(19) United States
(12) Patent Application Publication
(10) Pub. No.: US 2025/0144339 A1
(43) Pub. Date: May 8, 2025

(54) PATIENT INTERFACE

(71) Applicant: ResMed Pty Ltd, Bella Vista (AU)

(72) Inventors: Matthew EVES, Sydney (AU); Stewart Joseph WAGNER, Hawkesbury (AU); Maximilian Aji WIJOYOSENO, Singapore (SG); Chiew Khuen WONG, Sydney (AU)

(21) Appl. No.: 18/933,986

(22) Filed: Oct. 31, 2024

(30) Foreign Application Priority Data
Nov. 3, 2023 (AU) 2023903532

(51) Int. Cl. A61M 16/06

(52) U.S. Cl. CPC A61M 16/0605; A61M 16/0683; A61M 2202/0007; A61M 2202/02; A61M 2205/0216; A61M 2205/3331; A61M 2205/582; A61M 2209/088; A61M 2210/0618

(57) ABSTRACT

A patient interface for treatment of sleep disordered breathing, the patient interface comprising: a strap comprising: an anterior portion configured to be positioned anterior to the patient's face in use; and a seal-forming structure formed by or provided to the anterior portion; wherein the anterior portion of the strap is shaped to form a plenum chamber; wherein the strap forms a positioning and stabilising structure to provide a force to hold the seal-forming structure in a therapeutically effective position on the patient's head.

### CROSS-REFERENCE TO RELATED APPLICATIONS

This application claims priority to AU 2023903532 filed Nov. 3, 2023, the entire contents of which are hereby incorporated by reference.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem, e.g. see U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload, e.g. see U.S. Pat. No. 6,532,959 (Berthon-Jones).

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

Another form of therapy system is a mandibular repositioning device.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

Certain masks may cause some patients a feeling of claustrophobia, unease and/or may feel overly obtrusive.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

Consequently, some masks suffer from being obtrusive, aesthetically undesirable, costly, poorly fitting, difficult to use, and/or uncomfortable especially when worn for long or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy, especially if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications: WO 1998/004310; WO 2006/074513; WO 2010/135785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Inc. has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications describe examples of nasal pillows masks: International Patent Application WO 2004/073778 (describing amongst other things aspects of the SWIFT™ nasal pillows mask), US Patent Application 2009/0044808 (describing amongst other things aspects of the SWIFT™ LT nasal pillows mask); International Patent Applications WO 2005/063328 and WO 2006/130903 (describing amongst other things aspects of the MIRAGE LIBERTY™ full-face mask); International Patent Application WO 2009/052560 (describing amongst other things aspects of the SWIFT™ FX nasal pillows mask).

### 2.2.3.1.2 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

One technique is the use of adhesives, e.g. see US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 2.2.3.1.3 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

### 2.2.3.1.4 Pressurised Air Conduit Used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 2.2.3.6 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focussed airflow.

### 2.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogramalectrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold the seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises patient interface comprising a plenum chamber pressurisable to a therapeutic pressure of at least 4 cmH2O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has a hole therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface also comprises a positioning and stabilising structure to provide a force to hold the seal-forming structure in a therapeutically effective position on the patient's head.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

One form of the present technology comprises a patient interface for treatment of sleep disordered breathing, the patient interface comprising:
a strap configured to be worn by a patient in use, the strap comprising:
   an anterior portion configured to be positioned anterior to the patient's face in use; and
   a seal-forming structure formed by or provided to the anterior portion, the seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares;
   wherein the anterior portion of the strap is shaped to form a plenum chamber pressurisable to a therapeutic pressure of at least 4 cmH₂O above ambient air pressure, said plenum chamber including a plenum chamber inlet port sized and structured to receive a flow of air at the therapeutic pressure for breathing by a patient, and wherein the seal-forming structure is constructed and arranged to maintain said therapeutic pressure in the plenum chamber throughout the patient's respiratory cycle in use;
   wherein the strap forms a positioning and stabilising structure to provide a force to hold the seal-forming structure in a therapeutically effective position on the patient's head.

One form of the present technology comprises a patient interface for treatment of sleep disordered breathing, the patient interface comprising:
a plenum chamber pressurisable to a therapeutic pressure of at least 4 cmH2O above ambient air pressure, said plenum chamber including a plenum chamber inlet port sized and structured to receive a flow of air at the therapeutic pressure for breathing by a patient;
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a positioning and stabilising structure configured to provide a force to hold the seal-forming structure in a therapeutically effective position on the patient's head, the positioning and stabilising structure comprising a strap configured to be arranged, in use, so that an anterior portion of the strap is positioned anterior to the patient's face in use;
a vent to allow a continuous flow of gases exhaled by the patient from an interior of the plenum chamber to ambient, said vent being configured to maintain the therapeutic pressure in the plenum chamber in use;
wherein the anterior portion of the strap is shaped to form the plenum chamber;
wherein the seal-forming structure is formed by or provided to the anterior portion of the strap.
   In examples:
the anterior portion of the strap is thermoformed into a three-dimensional shape defining the plenum chamber;
the anterior portion of the strap comprises a concave portion defining the plenum chamber;
the seal-forming structure is attached to the anterior portion of the strap;
the seal-forming structure comprises a cushion attached to the anterior portion of the strap configured to sealingly engage the patient's face in use;
the cushion is formed from foam;
the anterior portion of the strap is shaped to form the seal-forming structure;
the anterior portion of the strap is thermoformed into a three-dimensional shape defining the plenum chamber and forming the seal-forming structure;
the anterior portion of the strap comprises a concave portion defining the plenum chamber and a convex portion provided around a periphery of the concave portion configured to engage the patient's face to form the seal-forming structure;
the strap is thicker in the convex portion than in the concave portion;
the strap comprises a plurality of layers;
the layers are bonded to each other;
the strap comprises at least one foam layer;
the strap comprises an inwardly facing layer configured to contact the patient's head in use and being formed from a textile material;
the strap comprises a resiliently compressible inner cushion layer;
the inner cushion layer is formed from foam;
the inner cushion layer comprises a varying thickness forming the shape of the seal-forming structure or of a portion of the strap to which the seal-forming structure is attached;
the strap comprises a semi-rigid support member;
the support member is in the form of a sheet formed from a plastic material;
the strap comprises a substantially inextensible layer;
the substantially inextensible layer is formed from webbing;
the substantially inextensible layer comprises a pair of high strength portions provided adjacent to the anterior portion on respective sides of the anterior portion and having a higher strength than adjacent portions of the substantially inextensible layer;
the high strength portions are provided at or proximate to an inferior edge of the strap;
the high strength portions may be formed from high strength webbing;
the strap comprises a resiliently compressible outer cushion layer;
the outer cushion layer is formed from foam;
the strap comprises an outwardly facing layer configured to face away from the patient's head in use and being formed from a textile material;
the anterior portion of the strap is thermoformed into a three-dimensional shape to form the plenum chamber;
the strap has been cut to a final shape by a cutting process which both cuts out the final shape of the strap and joins layers of the strap together at edges of the strap;
the cutting process is RF cutting;
the cutting process is performed after the anterior portion of the strap is thermoformed;
the strap comprises lateral portions on either lateral side of the anterior portion, the lateral portions being substantially flat at least at rest;
the strap comprises a thermoformed hinge portion between the anterior portion and the lateral portions of the strap;
the thermoformed hinge portion comprises a debossed portion of the strap;
the strap comprises a posterior portion configured to engage posterior surfaces of the patient's head and/or neck to hold the patient interface in an in use position;
the strap comprises a fastening portion at an end of the strap configured to attach to another portion of the strap to secure the strap around the patient's head;
the fastening portion comprises a hook material configured to attach to loop material on a surface of the strap;
each fastening portion comprises a plurality of thermoformed indexing portions;
the thermoformed indexing portions are embossed or debossed;
each fastening portion comprises at least one embossed indexing portion and at least one debossed indexing portion;
the at least one debossed indexing portion comprises a hook material attached thereto and configured to form a hook-and-loop connection with the at least one embossed indexing portion;
the patient interface comprises a buckle through which each of the fastening portions pass through and secure back to themselves;
the strap comprises an opening formed in the anterior portion of the strap connected to an inlet connector configured to fluidly connect to and receive the flow of air from an air circuit;
the inlet connector is connected to an inlet conduit configured to fluidly connect to and receive the flow of air from a conduit of the air circuit connected to a respiratory pressure therapy device generating the flow of air in use;
the inlet connector forms the plenum chamber inlet port; and/or.
the inlet connector further comprises the vent.

Another aspect of one form of the present technology is a patient interface that is moulded or otherwise constructed with a perimeter shape which is complementary to that of an intended wearer.

An aspect of one form of the present technology is a method of manufacturing apparatus.

Another aspect of one form of the present technology is a method of assembling a modular system comprising selecting a positioning and stabilising structure, and connecting the positioning and stabilising structure to either a first cushion or a second cushion.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 RESPIRATORY THERAPY SYSTEMS

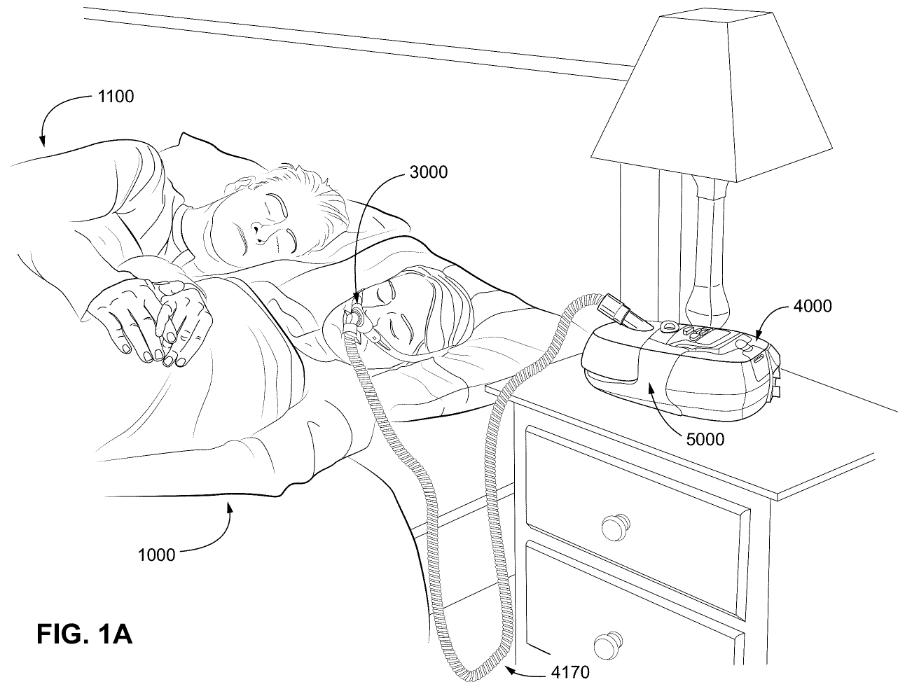

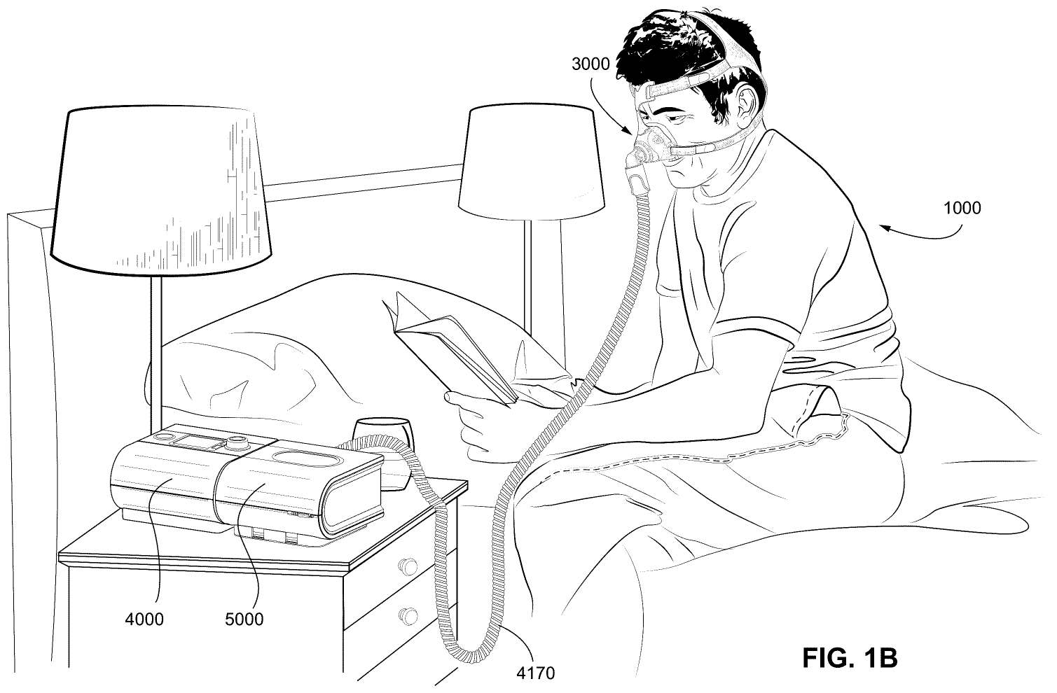

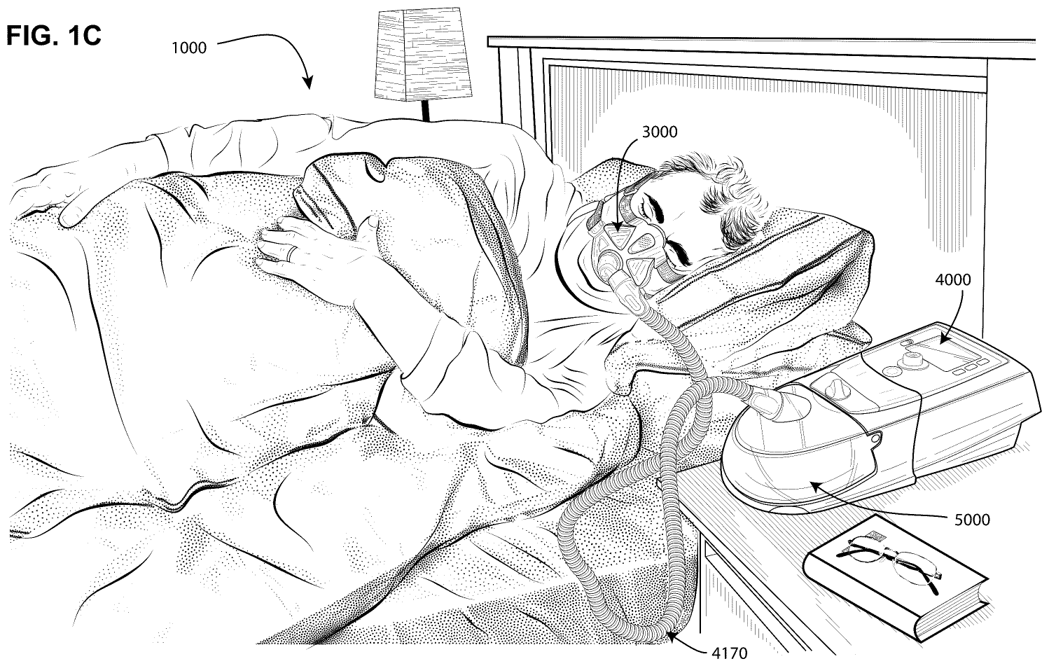

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

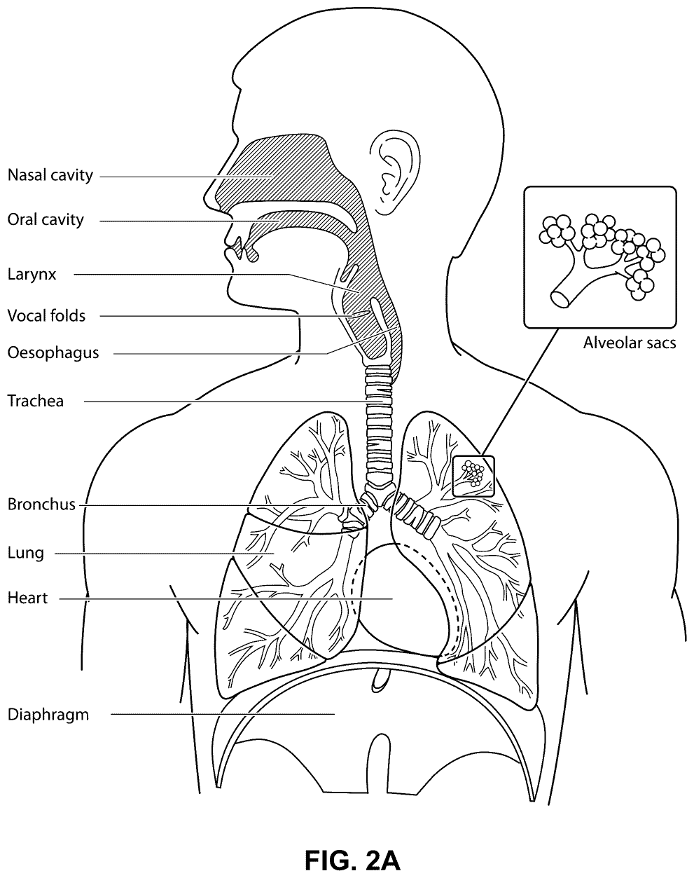

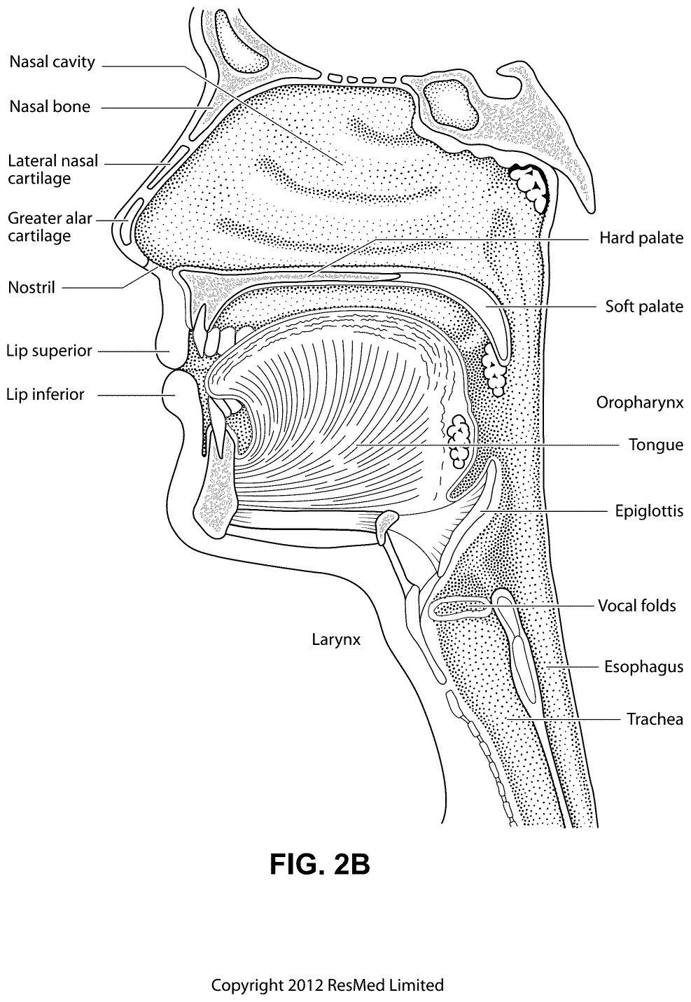

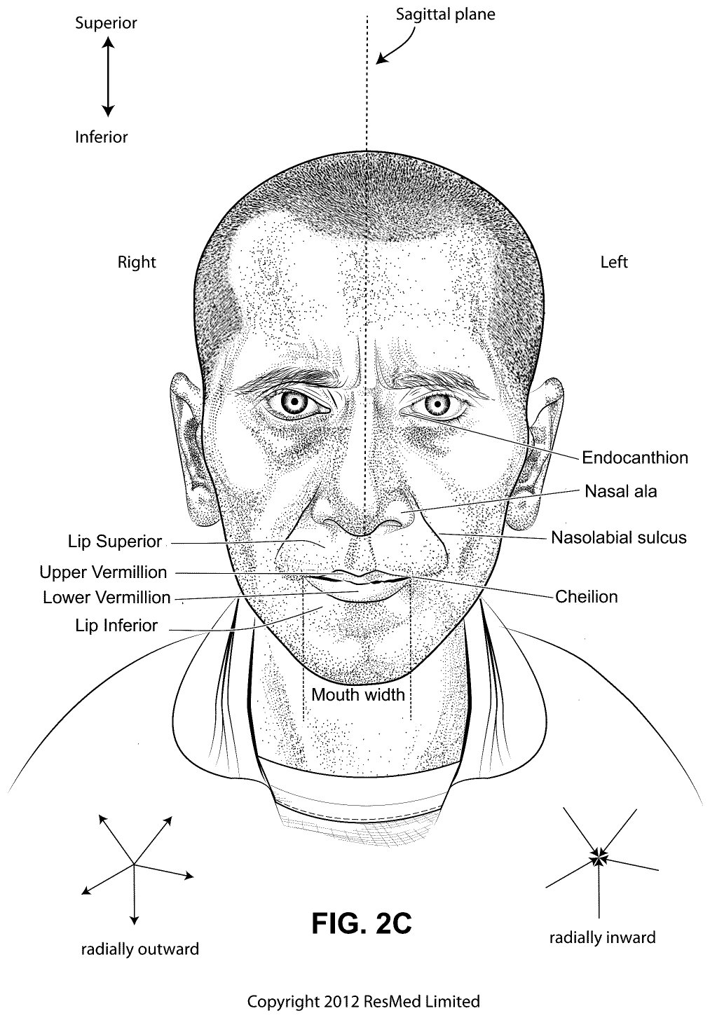

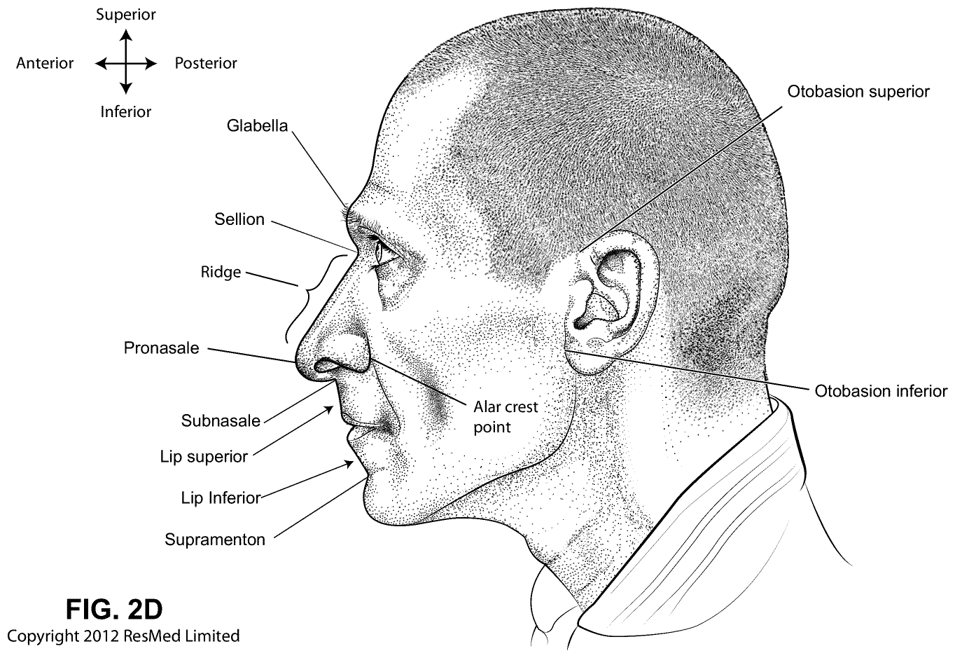

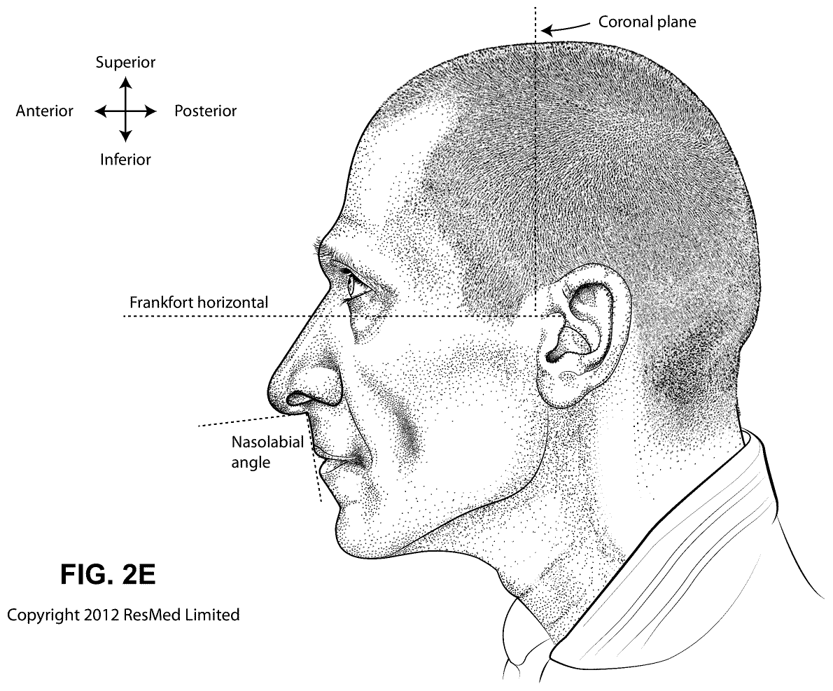

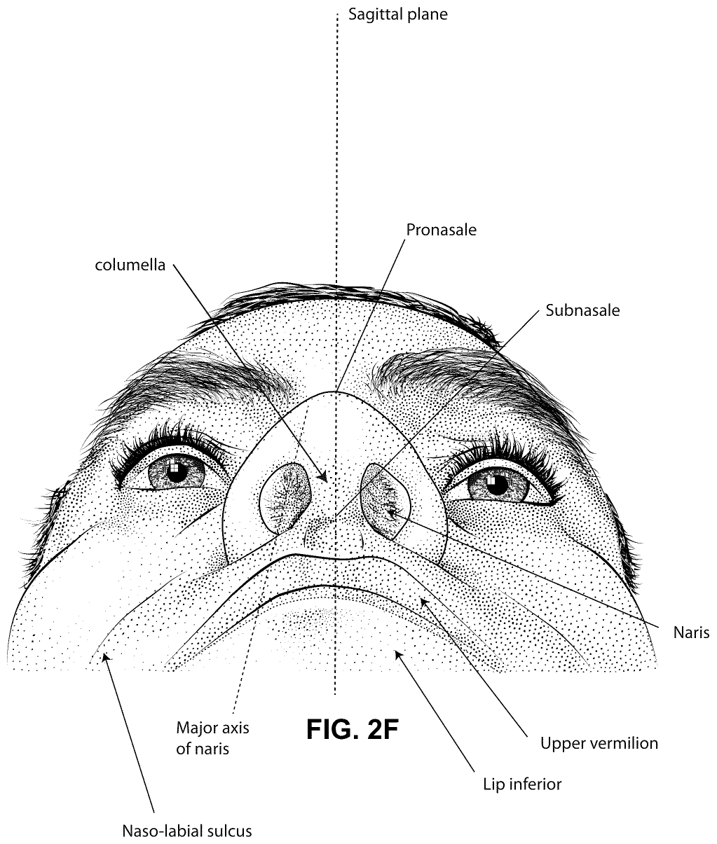

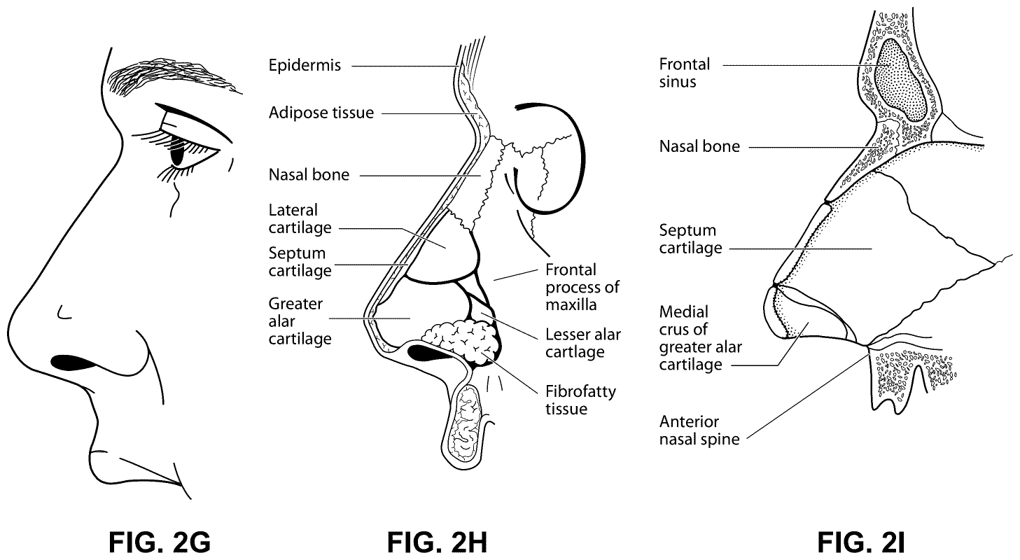

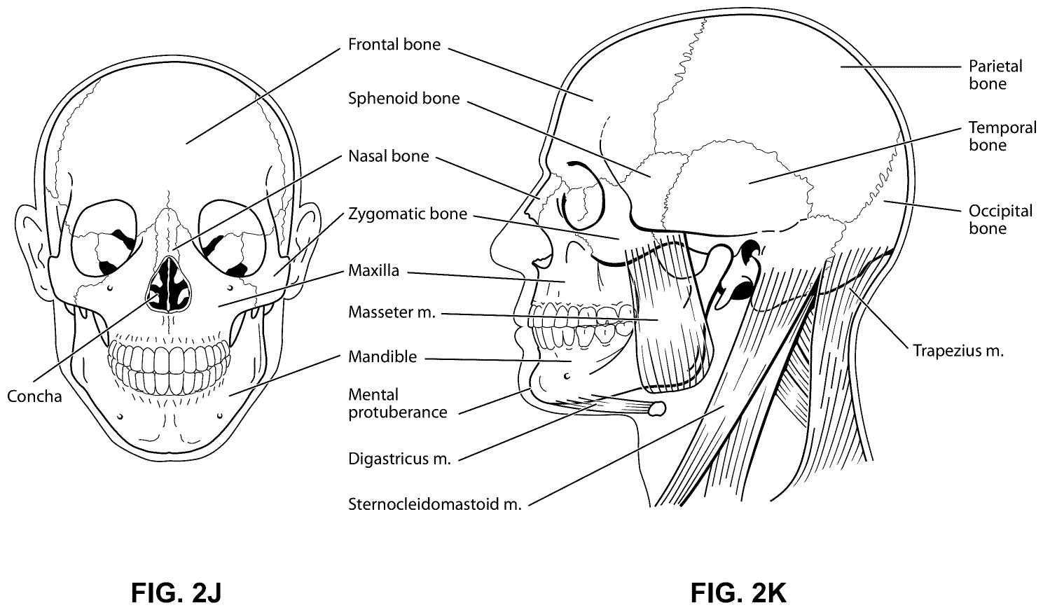

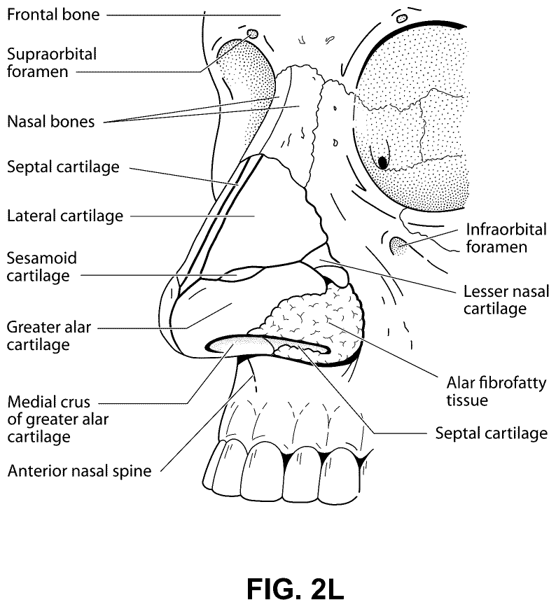

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoideopezius.

FIG. 2L shows an anterolateral view of a nose.

### 4.3 Patient Interface

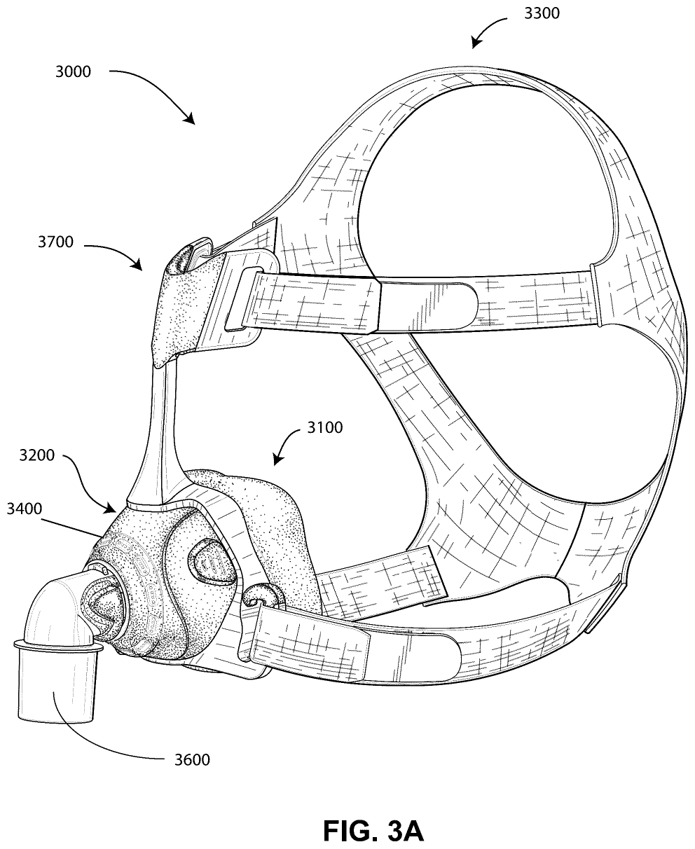

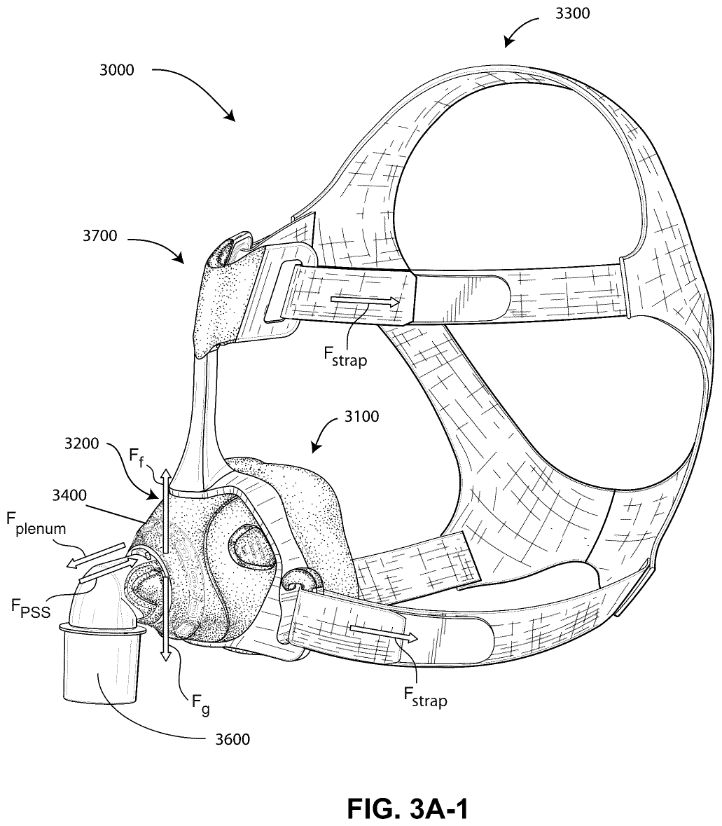

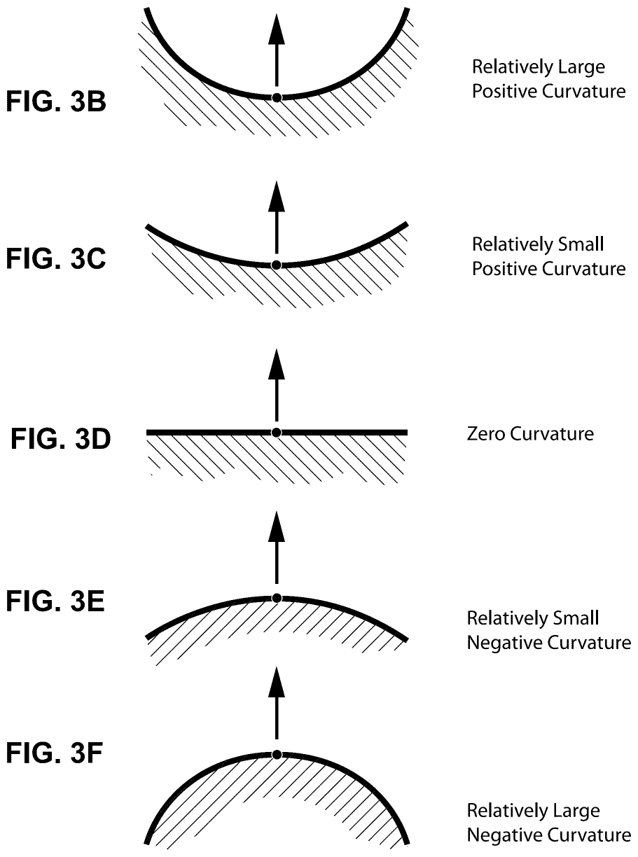

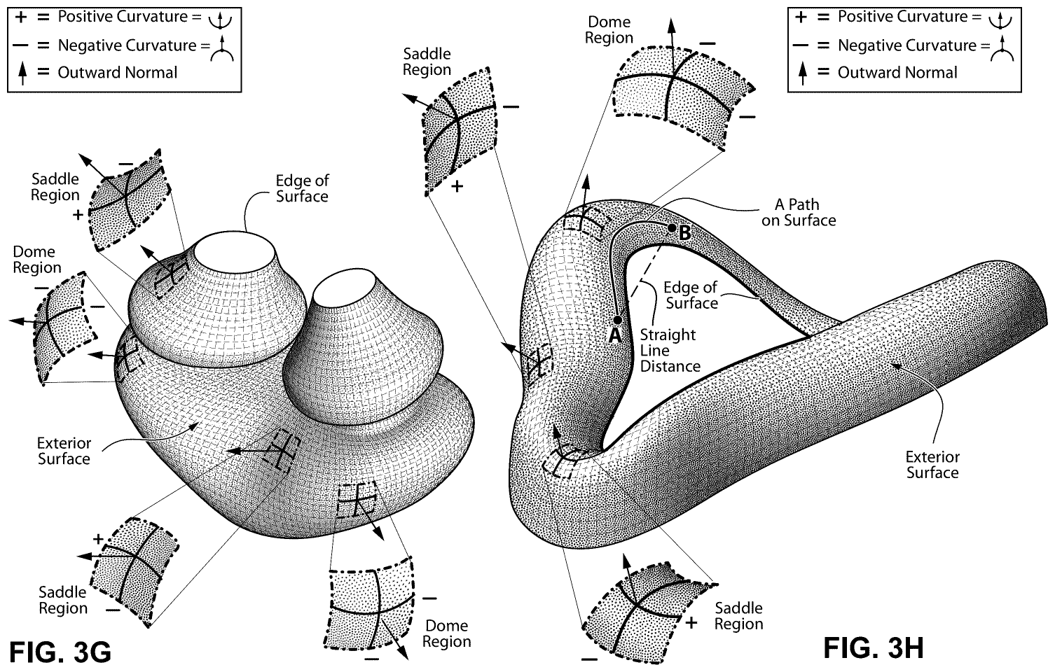

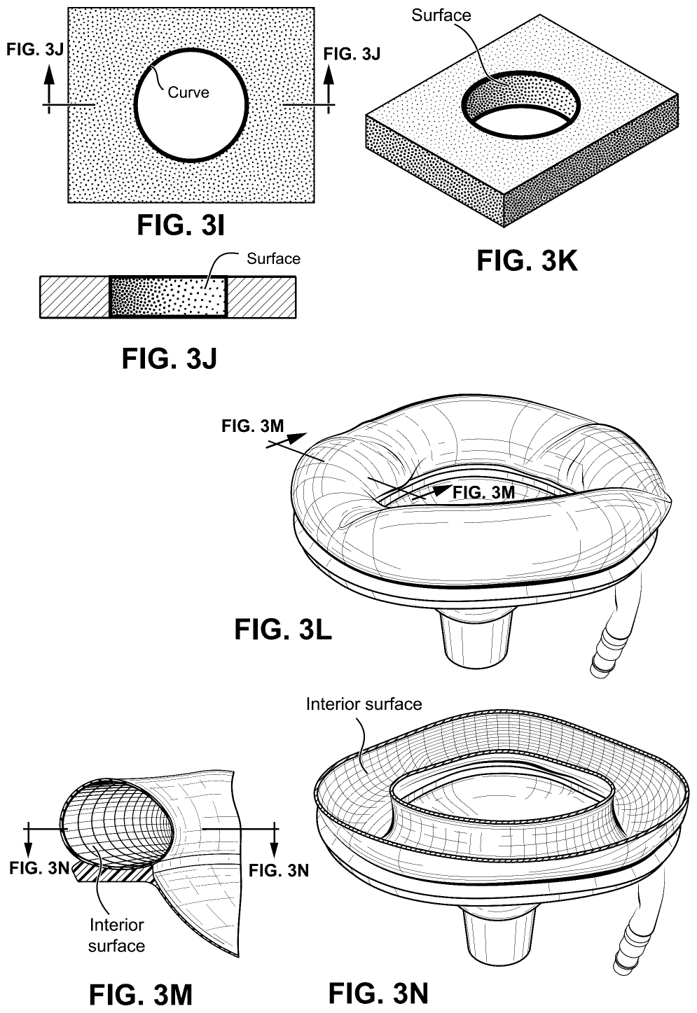

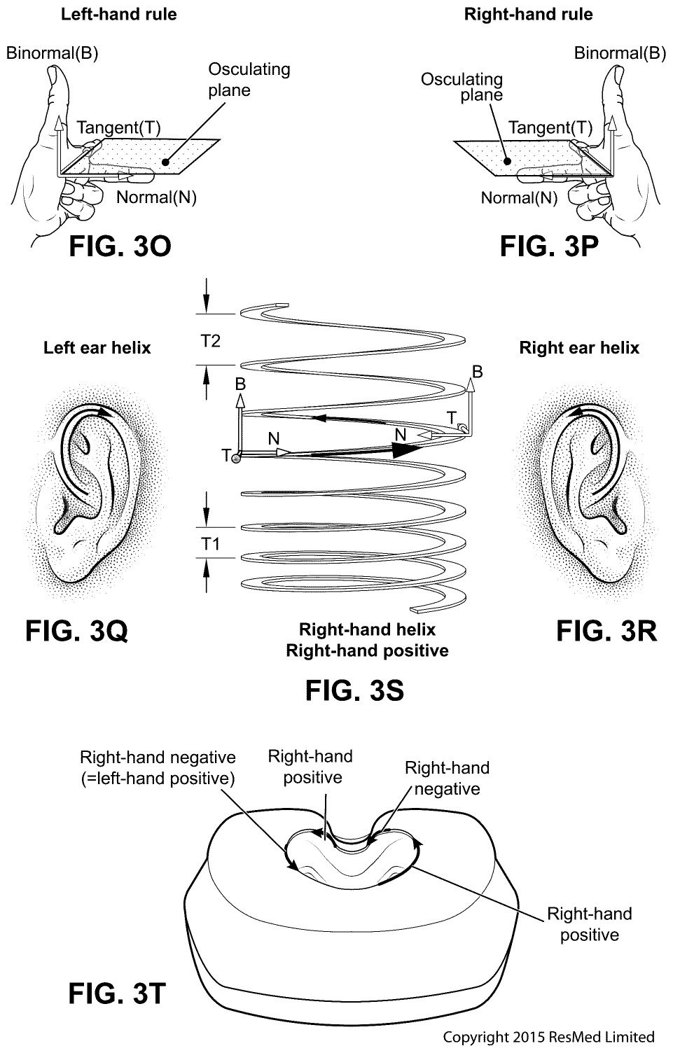

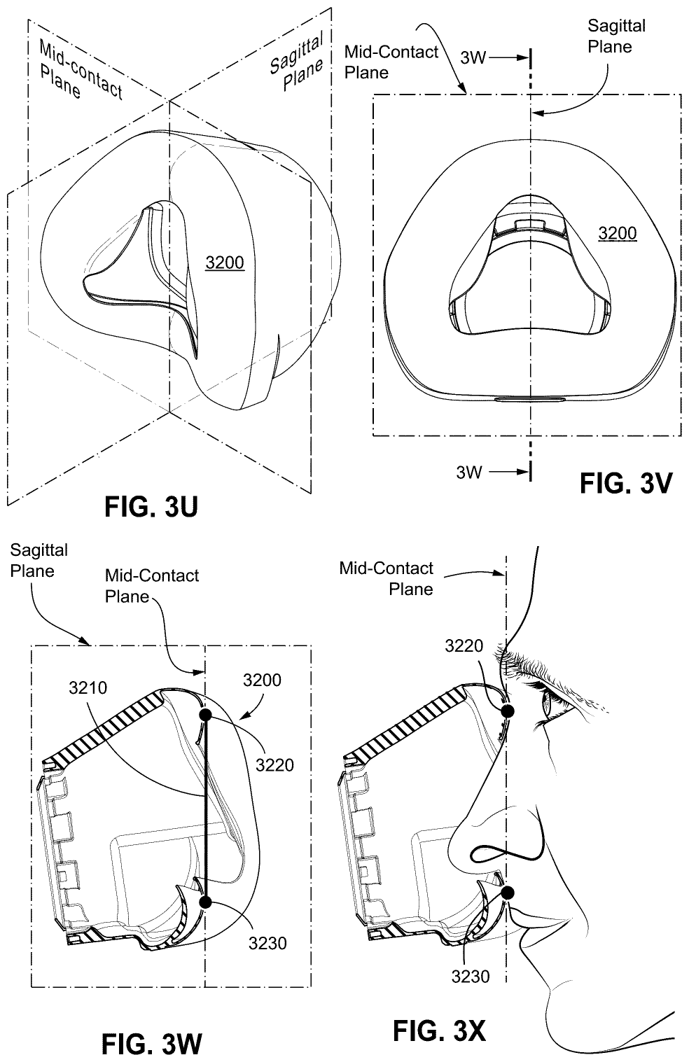

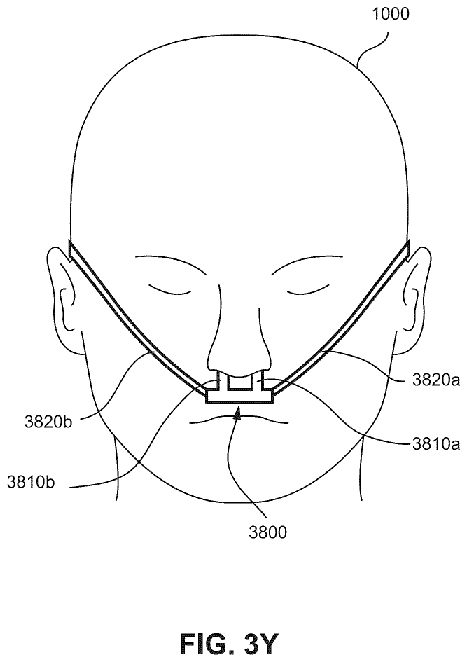

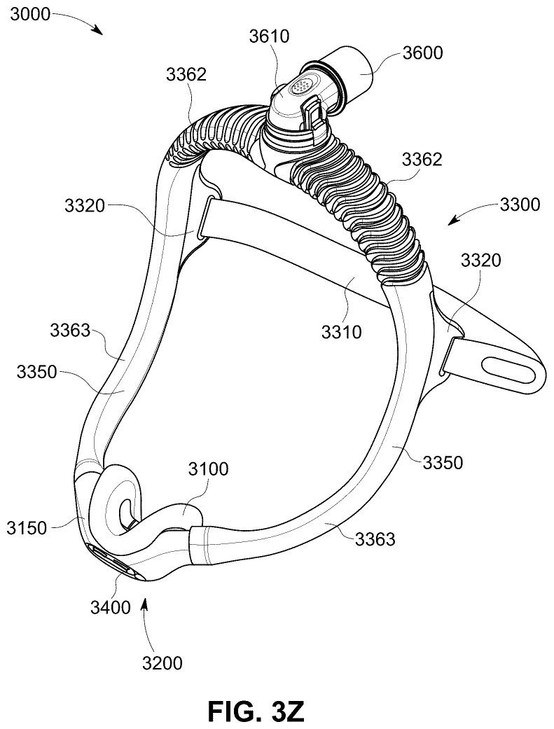

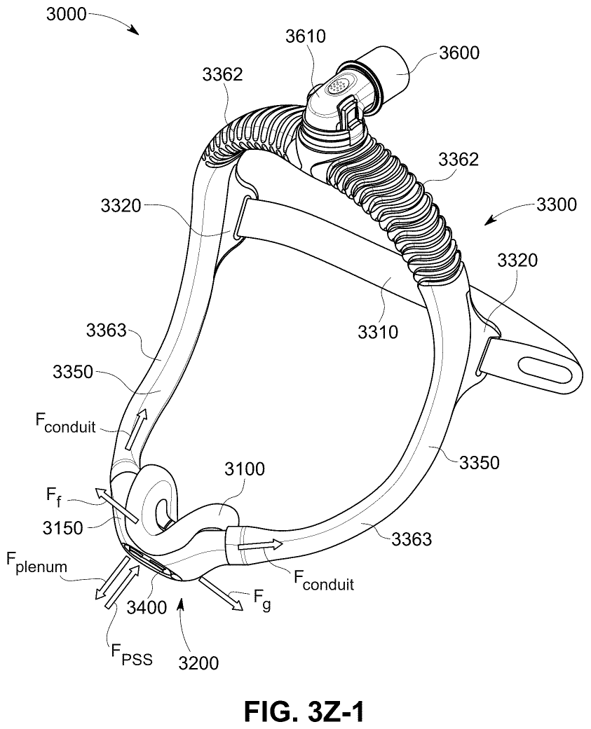

FIG. 3A shows a patient interface in the form of a nasal mask in accordance with one form of the present technology.

FIG. 3A-1 shows forces acting on the patient interface of FIG. 3A, while in use.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

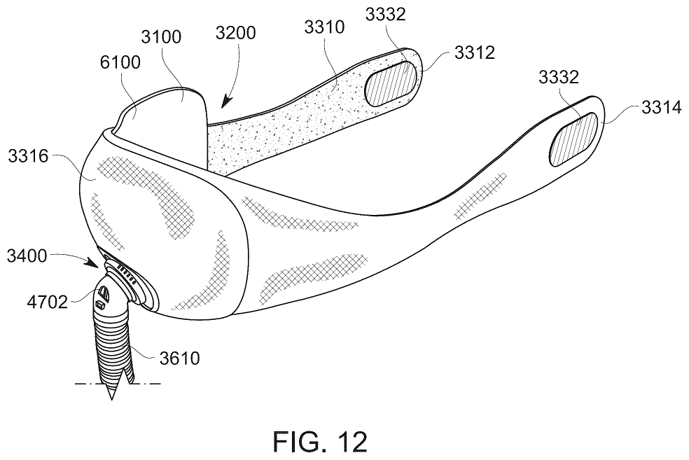

FIG. 3K shows a perspective view of the structure of FIG. 31, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 30 illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3220 sits approximately on the sellion, while the inferior point 3230 sits on the lip superior.

FIG. 3Y shows a patient interface in the form of a nasal cannula in accordance with one form of the present technology.

FIG. 3Z shows a patient interface having conduit headgear, in accordance with one form of the present technology.

FIG. 3Z-1 shows forces acting on the patient interface of FIG. 3Z, while in use.

### 4.4 Rpt Device

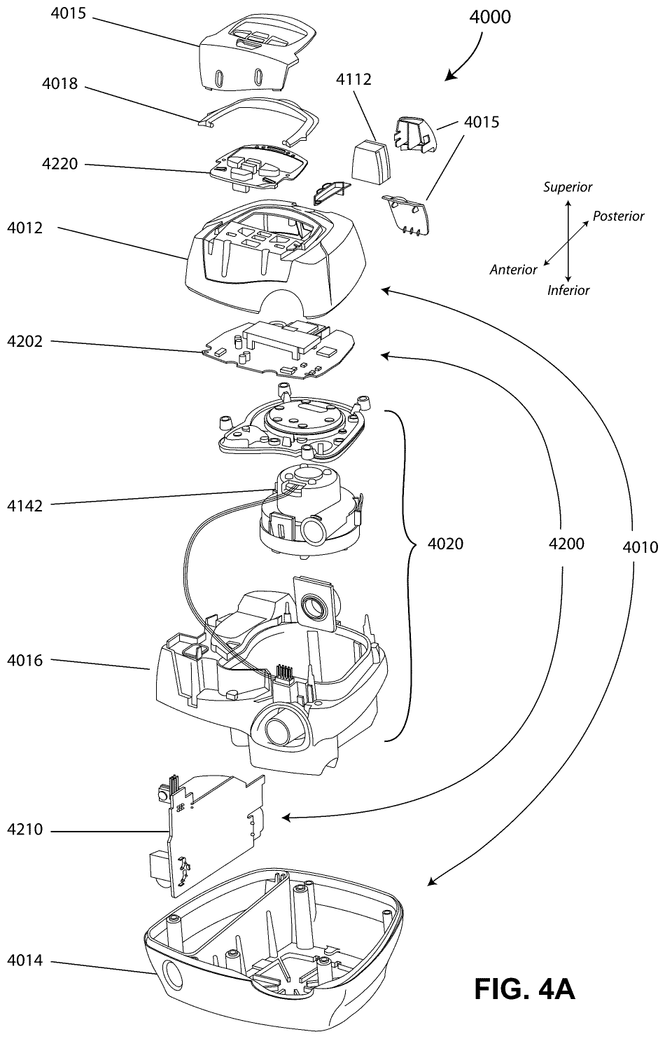

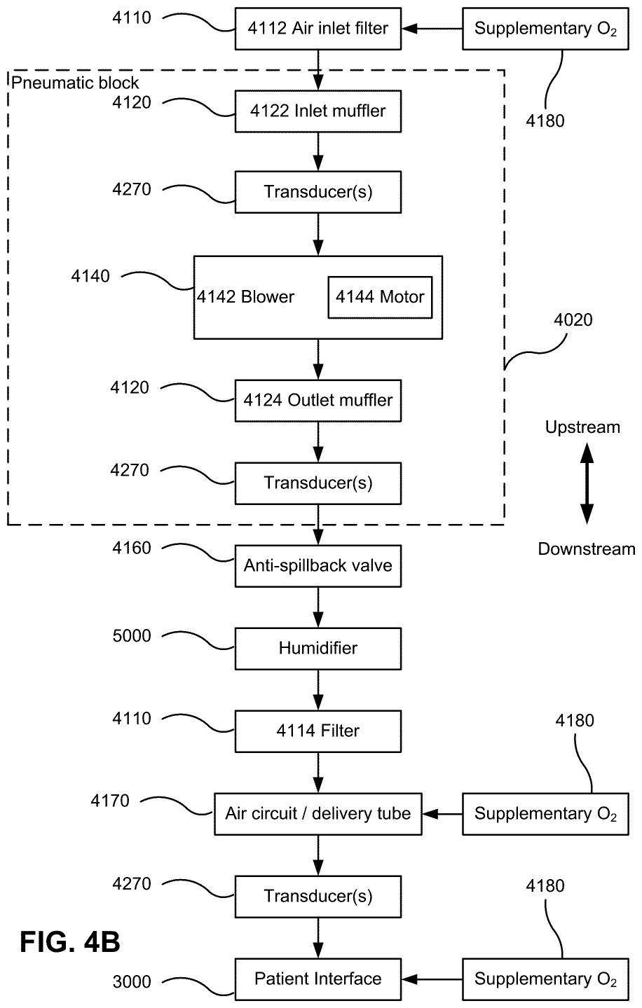

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 4.5 Humidifier

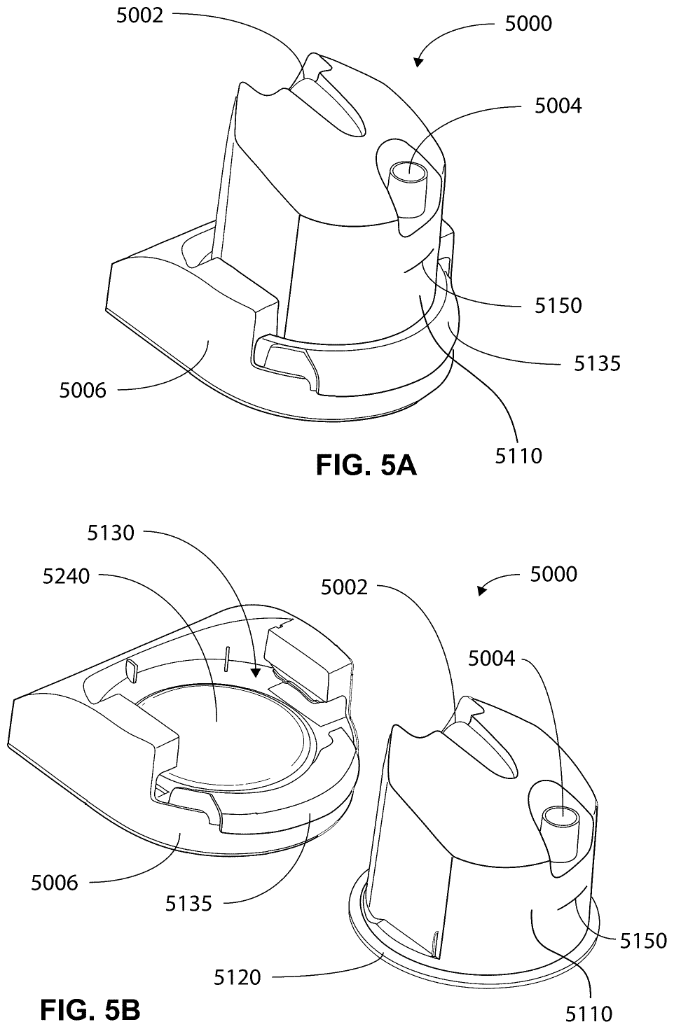

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 4.6 Breathing Waveforms

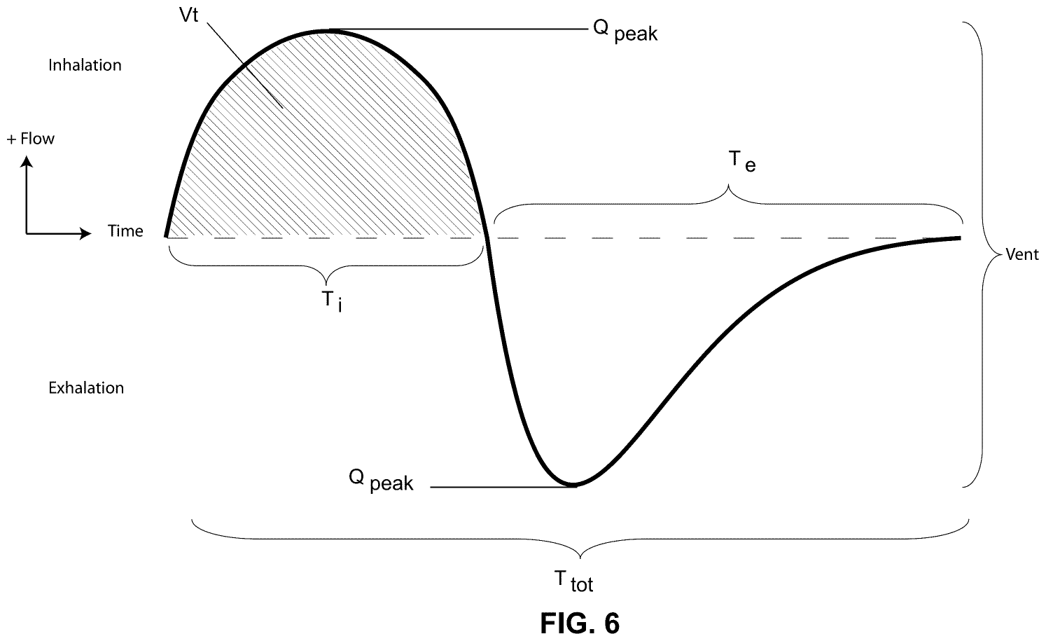

FIG. 6 shows a model typical breath waveform of a person while sleeping.

### 4.7 Further Examples of the Present Technology

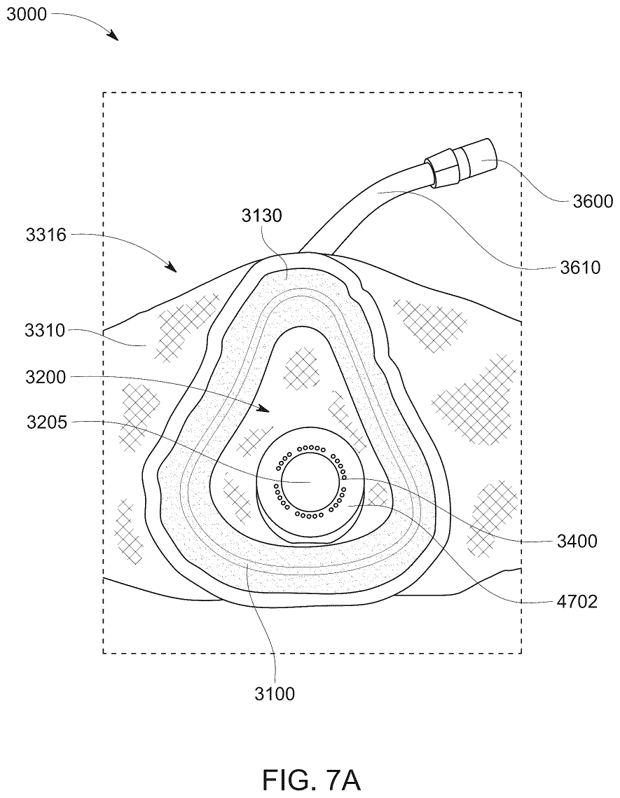

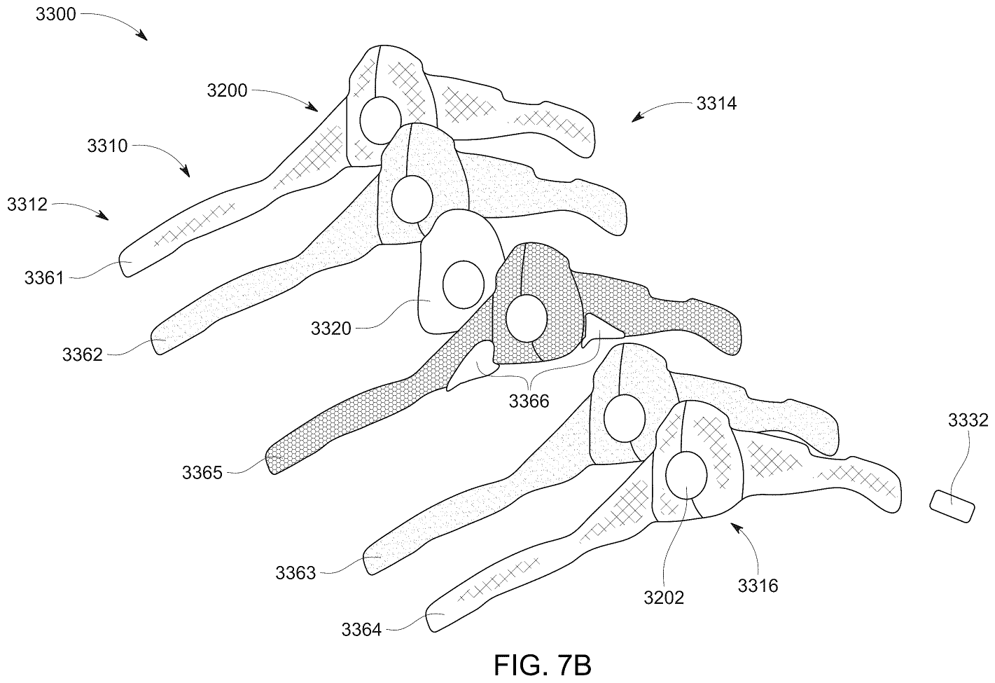

FIG. 7A shows a posterior view of a patient interface according to an example of the present technology.

FIG. 7B shows an exploded view of the patient interface of FIG. 7A.

FIG. 7C shows a schematic cross section view of a patient interface according to another example of the present technology.

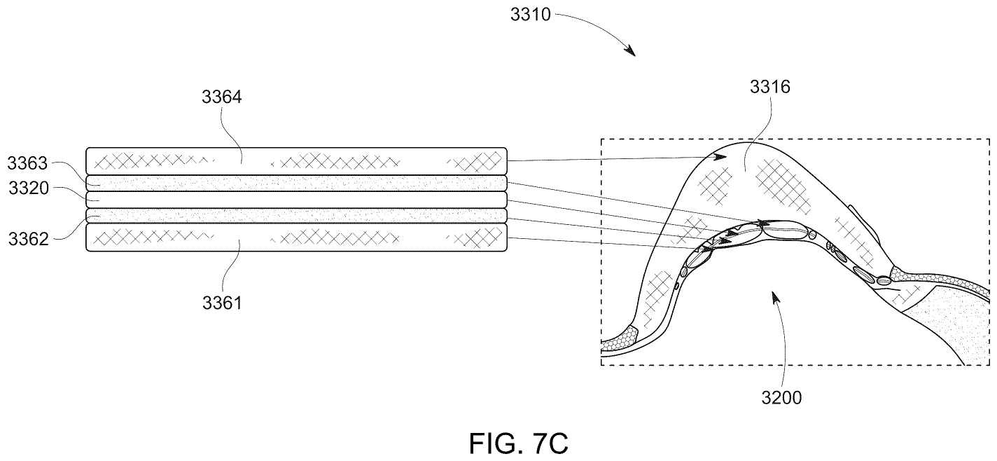

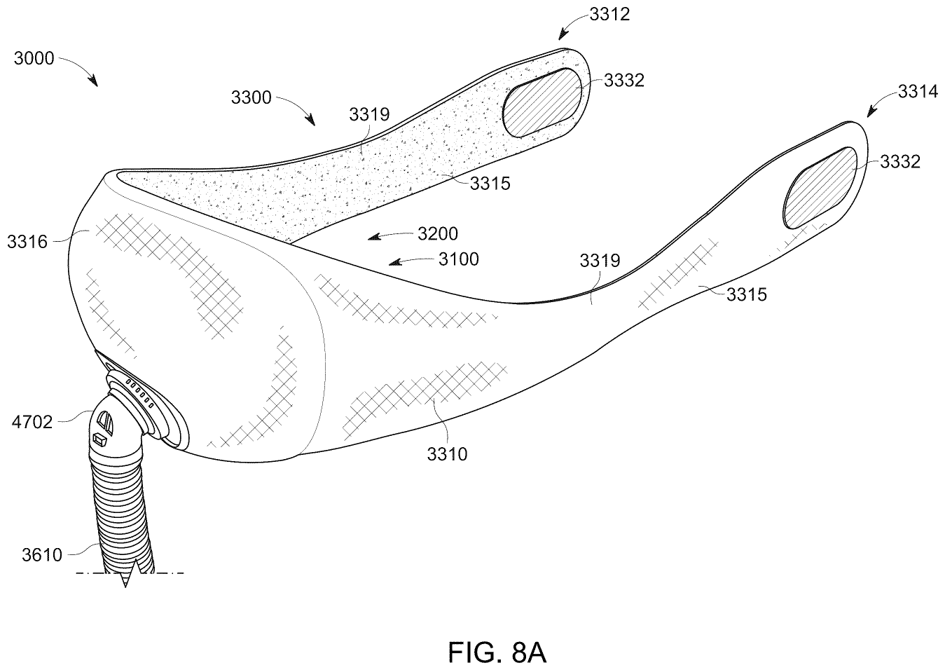

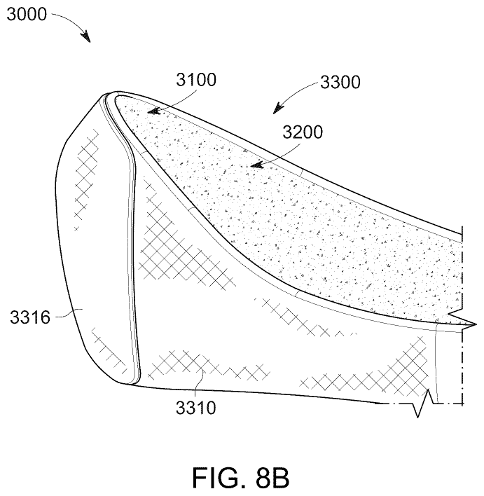

FIGS. 8A-8B show views of a patient interface according to another example of the present technology.

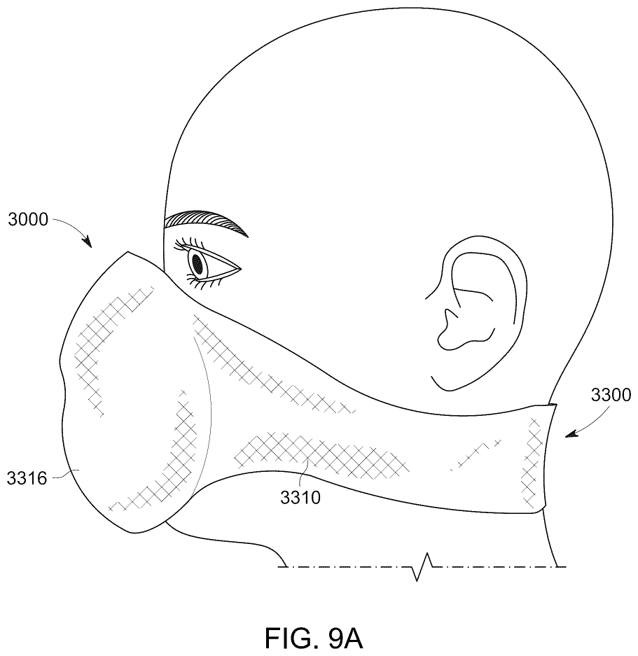

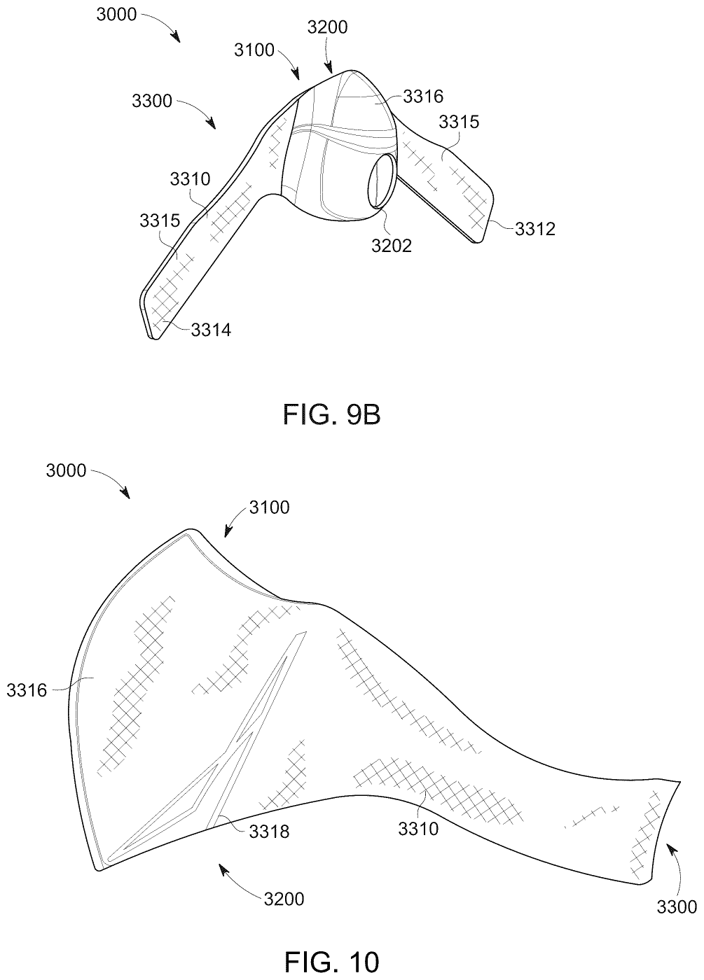

FIGS. 9A-B show views of a patient interface according to another example of the present technology.

FIG. 10 shows a lateral view of a patient interface according to another example of the present technology.

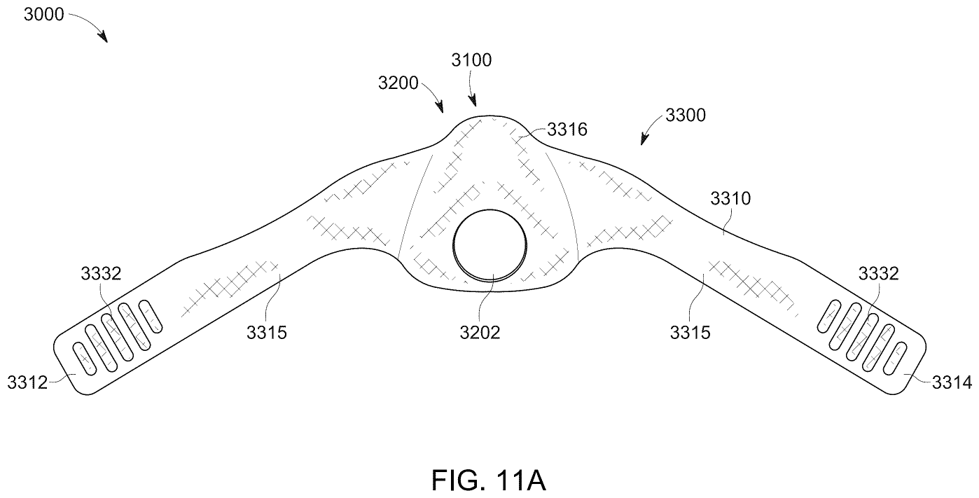

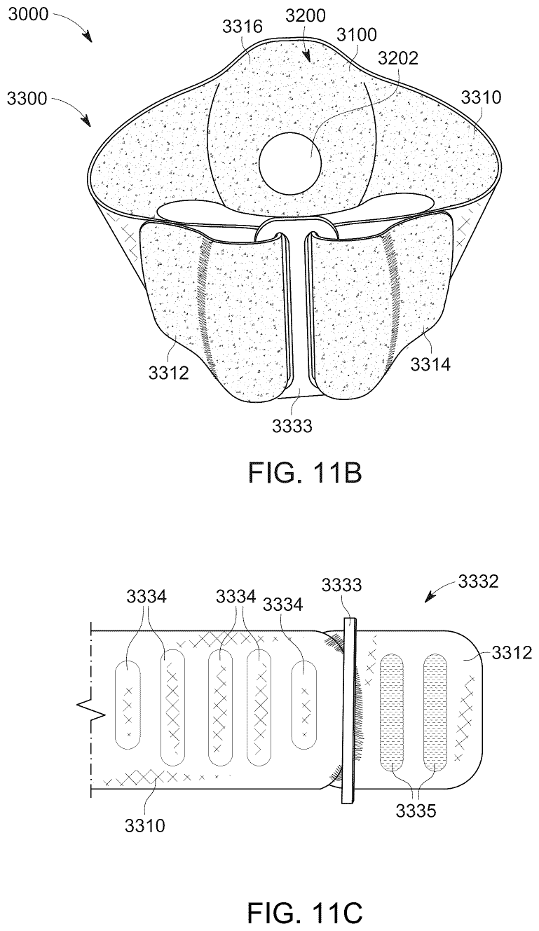

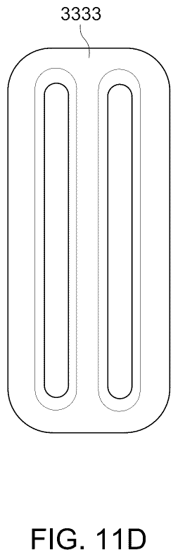

FIGS. 11A-11C show views of a patient interface according to another example of the present technology.

FIG. 11D shows a buckle according to another example of the present technology.

FIG. 12 shows a perspective view of a patient interface according to another example of the present technology.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 5.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 3Z, a non-invasive patient interface 3000 in accordance with another aspect of the present technology comprises the following functional aspects: a seal-forming structure 3100, a plenum chamber 3200, a positioning and stabilising structure 3300, a vent 3400 and one form of connection port 3600 for connection to an air circuit (such as the air circuit 4170 shown in FIGS. 1A-1C). The plenum chamber 3200 may be formed of one or more modular components (e.g., a cushion module 3150 together with the seal-forming structure 3100) in the sense that it or they can be replaced with different components, for example components of a different size.

An unsealed patient interface 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. This type of interface results in one or more gaps that are present in use by design (intentional) but they are typically not fixed in size such that they may vary unpredictably by movement during use. This can present a complex pneumatic variable for a respiratory therapy system when pneumatic control and/or assessment is implemented, unlike other types of mask-based respiratory therapy systems. The air to the nasal prongs may be delivered by one or more air supply lumens 3820a, 3820b that are coupled with the nasal cannula-type unsealed patient interface 3800. The lumens 3820a, 3820b lead from the nasal cannula-type unsealed patient interface 3800 to a respiratory therapy device via an air circuit. The unsealed patient interface 3800 is particularly suitable for delivery of flow therapies, in which the RPT device generates the flow of air at controlled flow rates rather than controlled pressures. The “vent” or gap at the unsealed patient interface 3800, through which excess airflow escapes to ambient, is the passage between the end of the prongs 3810a and 3810b of the nasal cannula-type unsealed patient interface 3800 via the patient's nares to atmosphere.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient, for example at least 2, 4, 6, 10, or 20 cmH2O with respect to ambient.

### 5.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 5.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 5.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 5.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 5.3.1.5 Forehead Region

In one form, the seal-forming structure that forms a seal in use on a forehead region of the patient's face. In such a form, the plenum chamber may cover the eyes in use.

### 5.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.1.7 Nose-Only Masks

In one form, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form, for example as shown in FIG. 3Z, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

In some forms, a nose-only mask may comprise nasal pillows, described above.

### 5.3.1.8 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 3100 configured to seal on the patient's face around the nose, below the mouth and over the bridge of the nose. A nose-and-mouth mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “nose-and-mouth cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

### 5.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

In some forms, the plenum chamber 3200 is constructed from a rigid material such as polycarbonate. The rigid material may provide support to the seal-forming structure.

In some forms, the plenum chamber 3200 is constructed from a flexible material (e.g., constructed from a soft, flexible, resilient material like silicone, textile, foam, etc.). For example, in examples then may be formed from a material which has a Young's modulus of 0.4 GPa or lower, for example foam. In some forms of the technology the plenum chamber 3200 may be made from a material having Young's modulus of 0.1 GPa or lower, for example rubber. In other forms of the technology the plenum chamber 3200 may be made from a material having a Young's modulus of 0.7 MPa or less, for example between 0.7 MPa and 0.3 MPa. An example of such a material is silicone.

The plenum chamber 3200 may comprise at least one plenum chamber inlet port. The plenum chamber inlet port may be configured to allow a flow of air or breathable gas at the therapeutic pressure into the plenum chamber in use. In some examples a patient interface 3000 may comprise multiple plenum chamber inlet ports. In some forms, a plenum chamber inlet port may also allow flow out of the plenum chamber 3200, for example towards a vent 3400 of the patient interface 3000 during the patient's exhalation phase of the respiratory cycle, or for example in the absence of a flow of air or breathable gas to the plenum chamber inlet port.

### 5.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position. Examples of a positioning and stabilising structure may be shown in FIGS. 3A and 3A-1.

In one form the positioning and stabilising structure 3300 provides a retention force at least sufficient to overcome the effect of the positive pressure in the plenum chamber 3200 to lift off the face (i.e., Fₚₗₑₙᵤₘ).

In one form the positioning and stabilising structure 3300 provides a retention force to overcome the effect of the gravitational force on the patient interface 3000.

With continued reference to FIG. 3A-1, the positioning and stabilising structure 3300 provides a force F_{PSS} that assists in maintaining the plenum chamber 3200 in the sealing position on the patient's face. The positioning and stabilising force F_{PSS} may be the resultant force from the various forces of the different elements of the positioning and stabilising structure 3300. For example, headgear straps may individually provide a strap force Fₛₜᵣₐₚ in order to hold the seal-forming structure 3100 against the patient's face. The force Fₛₜᵣₐₚ may also be directed at least partially in the superior direction in order to overcome the gravitational force F_{g}. The gravitational force F_{g} may be specifically shown for the seal-forming structure 3100 and the plenum chamber 3200, but gravity would act on the entirely of the patient interface 3000 (i.e., in the same direction as the illustrated gravitational force F_{g}).

The gravitational force F_{g} may be opposed by a frictional force F_{f}, which may act in a direction directly opposite of the gravitational force F_{g}. As gravity pulls the seal-forming structure 3100 and the plenum chamber 3200 in the inferior direction (as viewed in FIG. 3A-1), the frictional force Fr would act in the superior direction (e.g., against a patient's face). For example, the patient may experience the frictional force Fr against his lip superior (and/or other surfaces of the patient's face in contact with the seal-forming structure 3100) in order to oppose the motion in the inferior direction (which may help to stabilising the cushion in place). Although the frictional force Fr is shown specifically opposing the gravitational force F_{g} of the seal-forming structure 3100 and the plenum chamber 3200, components of an overall frictional force (not shown) would also oppose the gravitational force F_{g} associated with the positioning and stabilising structure 3300 and any other portions of the patient interface 3000. A force of friction can act along any place where the patient interface 3000 contacts the patient's skin (or hair). The frictional force Fr extends in the opposite direction of the gravitational force F_{g} and along the patient's skin (or hair). In some forms the gravitational force F_{g} may also be countered by vertical components of the reaction force from the patient's face acting on the seal-forming structure 3100, for example at the nose ridge and chin regions of the patient's face, for example.

In some forms, the sum of the various forces may equal zero so that the patient interface 3000 is at equilibrium (e.g., not moving along the patient's face while in use). Specifically, the gravitational force F_{g} and the blowout force Fₚₗₑₙᵤₘ tend to move the seal-forming structure 3100 away from the desired sealing position. The positioning and stabilising force F_{PSS} is applied in order to counteract the gravitational force F_{g} and the blowout force Fₚₗₑₙᵤₘ (as well as any frictional forces F_{f}) and keep the seal-forming structure 3100 properly situated. Although the positioning and stabilising force F_{PSS} may exceed the sum of the gravitational force F_{g} and the blowout force Fₚₗₑₙᵤₘ (with any additional positioning and stabilising force F_{PSS} being balanced by reaction force from the patient's head acting on the portions of patient interface 3000) and still maintain the seal-forming structure 3100 in an appropriate scaling position, patient comfort may be sacrificed. Maximum patient comfort may be achieved when the net force on the patient interface 3000 is zero and the positioning and stabilising force F_{PSS} is exactly strong enough to achieve this. In some examples the positioning and stabilising structure 3300 may be adjustable such that when fitted the positioning and stabilising force F_{PSS} is greater than required to exactly balance the gravitational force F_{g} and the blowout force Fₚₗₑₙᵤₘ to hold the patient interface 3000 against the patient's head tightly enough that disruptive forces which may be experienced in use (such as tube drag or lateral shunting of the plenum chamber 3200 during side sleeping) do not disrupt the seal. As described below, various positions of the patient's head while using the patient interface 3000 may determine the positioning and stabilising force F_{PSS} necessary to achieve equilibrium.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilising structure 3300, each being configured to provide a retaining force to correspond to a different size and/or shape range. For example the system may comprise one form of positioning and stabilising structure 3300 suitable for a large sized head, but not a small sized head, and another. suitable for a small sized head, but not a large sized head.

### 5.3.3.1 Conduit Headgear

### 5.3.3.1.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3Z, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

### 5.3.3.2 Headgear Straps

In some forms, the positioning and stabilising structure 3300 may include headgear comprising at least one strap which may be worn by the patient in order to assist in properly orienting the seal-forming structure 3100 against the patient's face (e.g., in order to limit or prevent leaks).

As described above, some forms of the headgear may be constructed from a textile material, which may be comfortable against the patient's skin. The textile may be flexible in order to conform to a variety of facial contours. Although the textile may include rigidisers along a selected length, which may limit bending, flexing, and/or stretching of the headgear.

In certain forms, the headgear may be at least partially extensible. For example, the headgear may include elastic, or a similar extensible material. For example, the entire headgear may be extensible or selected portions may be extensible (or more extensible than surrounding portions). This may allow the headgear to stretch while under tension, which may assist in providing a sealing force for the seal-forming structure 3100.

### 5.3.3.3 Single Strap Patient Interface

In some forms of the present technology there is provided a patient interface 3000 comprising a plenum chamber 3200, a seal-forming structure 3100, a positioning and stabilising structure 3300, and a vent 3400. FIGS. 7A-12 show some such exemplary patient interfaces 3000. In these examples, the positioning and stabilising structure 3300 comprises a strap 3310 configured to be arranged, in use, so that an anterior portion 3316 of the strap 3310 is positioned anterior to the patient's face in use.

### 5.3.3.3.1 Single Strap Configurations

The strap 3310 may take different forms. In the examples shown in FIGS. 7A-7C, 8A-8B, 9A-9B and 11A-11D and 12, the straps 3310 comprise a posterior portion configured to engage posterior surfaces of the patient's head and/or neck to hold the patient interface 3000 in an in-use position. In these examples, when worn, the strap 3310 encircles the patient's head and neck. The strap 3310 consequently has the anterior portion 3316 positioned anterior to the patient's face and a posterior portion positioned on a posterior side of the patient's head and overlying the posterior of the patient's head, for example the posterior of the patient's head and/or neck. The strap 3310 in this form also has lateral portions 3315 that are positioned on the sides of the patient's head and overlie the sides of the patient's head. The anterior, posterior and lateral portions of the strap 3310 are connected together in a loop when worn. The lateral portions 3315 may comprise narrow portions 3319. The narrow portions 3319 may be formed by a portion of each lateral portion 3315 having a reduced height in the superior-inferior directions in comparison to the strap 3310 proximate the anterior portion 3316 and/or proximate the first end 3312 and second end 3314. Each narrow portion 3319 may be located between the anterior portion 3316 and a respective one of the first end 3312 and second end 3314. The narrow portions 3319 may help the strap 3310 avoid the ears and/or reduce bulk on the sides of the patient's neck.

In some examples, such as are shown in FIGS. 7A-12, the positioning and stabilising structure 3300 consists of only a single strap 3310. That is, there are no other straps that comprise part of the positioning and stabilising structure 3300 and the strap 3310 is capable of maintaining the seal-forming structure 3100 in therapeutically effective (e.g. sealed) position on its own. Some conventional positioning and stabilising structures for patient interfaces take the form of headgear that consists of multiple straps attached together in different directions because each strap is intended to sit on a different part of the patient's head. Such headgear assemblies can be complicated for a patient to arrange and put on their head, particularly in the dark and if the patient is tired or lacks dexterity. Forms of the technology described herein that consist of a single strap 3310 are simpler and more intuitive for a patient to put on because only a single strap needs to be handled and oriented correctly. This may promote the patient to use the patient interface and conform with their therapy prescription.

The strap 3310 may be formed from one or more materials. In some forms in which the strap 3310 is formed from a plurality of materials, the materials may be blended, for example woven, together to form a single blended material. In other forms in which the strap 3310 is formed from a plurality of materials, the strap may comprise a plurality of portions, each formed from a different material, that are connected together to form the strap 3310.

With reference to FIGS. 7A-7C and 8A-8B for example, the strap 3310 may comprise a fastening portion 3332 at an end of the strap configured to attach to another portion of the strap to secure the strap around the patient's head. In certain forms of the technology, the strap 3310 comprises a first end 3312 and a second end 3314. The first and second ends 3312 and 3314 are separated by the length of the strap. The first end 3312 may be configured to be secured to the second end 3314 to form a loop, and may comprise a fastening portion 3332. In some forms, hook-and-loop material may be used to effect the securing of the first end 3312 to the second end 3314. For example, a first hook-and-loop material segment (which may be the hook or loop segment) may be provided to the first end 3312 and a second hook-and-loop material segment (which is the other of the hook or loop segment) may be provided to the second end 3312 on the other side of the strap 3310. The hook segment may be placed on a side of the strap 3310 facing away from the patient in use to reduce the level of discomfort experienced by the patient. In other forms, other connectors for securing the two ends of the strap 3310 together may be used, for example poppers, domes, clips and the like. A strap 3310 with detachable ends may be relatively easy for the patient to put on. A hook-and-loop securement mechanism may also be advantageous as it allows the effective length of the strap 3310 to be adjusted finely in order to fit the patient's head. In forms of the technology in which other connectors are used (e.g. poppers, domes and clips), multiple instances of the connectors may be provided to the strap 3310 in order to allow adjustment of the effective length of the strap 3310.

In some examples, the fastening portion 3332 may comprise a hook material configured to attach to complementary (e.g. loop) material on a surface of the strap 3310. In some examples the strap 3310 may comprise a fastening portion 3332 on each end of the strap. Each fastening portion 3332 may be configured to connect to the other fastening portion 3332. Alternatively, each fastening portion 3332 may be configured to connect to material forming the strap 3310, such as an outermost layer of the strap 3310. With reference to FIGS. 7A-7C and 8A-8B for example, the strap 3310 comprises a fastening portion 3332 at each of the first end 3312 and the second end 3314. More generally, the fastening portion 3332 may be formed from hook material configured to form a hook-and-loop connection with the surface of the strap 3310 at or proximate the second end 3314 or with another (e.g. complementary) fastening portion 3332 at the second end 3314, such as a fastening portion 3332 formed from a loop material.

In other forms of the technology, the strap 3310 may be formed as a continuous loop without ends. In such a form, the strap 3310 may be donned by being pulled down over the top of the head.

In some examples, such as the example shown in FIGS. 8A and 8B, the anterior portion 3316 may be approximately mid-way between the first end 3312 and the second end 3314. When the strap 3310 is worn by the patient, the anterior portion 3316 may be approximately positioned on the opposite side of the patient's head from the region at which the first end 3312 and the second end 3314 overlap or otherwise join to each other. In certain forms, the strap 3310 is configured to be arranged, when worn, so that the anterior portion 3316 of the strap 3310 is positioned at the patient's airways and the posterior portion of the strap 3310 overlying the posterior region of the patient's neck comprises the first end 3312 and/or second end 3314. In other forms, the anterior portion 3316 may be positioned in a different location with respect to a circumference around the patient's head. Arranging the strap 3310 with ends that connect together around the back of the head with the anterior portion 3316 directly in front of the patient may be intuitive for the patient and also moves the ends of the strap 3310, which may be more prone to rub on the patient's skin around the back of the head away from the sensitive areas of the patient's face.

### 5.3.3.3.2 Inlet Connector

As shown in FIGS. 7B, 9B, 11A and 11B for example, the strap 3310 may comprise an opening 3202 formed in the anterior portion 3316 of the strap 3310. In use the opening 3202 may be connected to an inlet connector 4702 (shown in FIGS. 7A, 8A and 12 for example) configured to fluidly connect to and receive a flow of air from an air circuit 4170. The inlet connector 4702 may be connected to an inlet conduit 3610 (e.g. a short tube) configured to fluidly connect to and receive the flow of air from a conduit of the air circuit 4170 connected to a respiratory pressure therapy device 4000 generating the flow of air in use. Alternatively the inlet connector 4702 may be structured to connect directly to a conduit of an air circuit 4170.

The inlet connector 4702 may form a plenum chamber inlet port 3205, as illustrated in FIG. 7A. The plenum chamber inlet port 3205 may be sized and structured to receive a flow of air at the therapeutic pressure for breathing by a patient. Also as shown in FIGS. 7A, 8A and 12 for example the inlet connector 4702 may comprise a vent 3400. The inlet connector 4702 may comprise a plurality of holes forming the vent 3400. In other examples the vent 3400 may be provided separately, for example in the anterior portion 3316 of the strap 3310.

### 5.3.3.3.3 Strap Forming Plenum Chamber

In some forms of the present technology, such as those shown in FIGS. 7A-7C, 8A-8B, 9A-9B, 10 and 11A-11B, the anterior portion 3316 of the strap 3310 may be shaped to form the plenum chamber 3200. The anterior portion 3316 may define the plenum chamber 3200. For example, a posterior surface of the anterior portion 3316 of the strap 3310 may at least partially define the plenum chamber 3200. It is to be understood that an anterior portion 3316 of a strap 3310 may be said to define the plenum chamber 3200 even if the plenum chamber 3200 is partially formed by other components, such as an inlet connector 4702 or even the patient's face. The strap 3310 or at least the anterior portion 3316 thereof may be constructed to be airtight. Alternatively, in some examples the anterior portion 3316 may be constructed to allow for a controlled leak though the material forming the anterior portion 3316, for the purpose of forming a vent 3400 through the material forming the anterior portion 3316. The seal-forming structure 3100 may be formed by or provided to the anterior portion 3316 of the strap 3310.

In these examples, the plenum chamber 3200 may be pressurisable to a therapeutic pressure of at least 4 cmH₂O above ambient air pressure and the plenum chamber 3200 may include a plenum chamber inlet port 3205 sized and structured to receive a flow of air at the therapeutic pressure for breathing by a patient. The seal-forming structure 3100 may be constructed and arranged to form a seal with a region of the patient's face surrounding an entrance to the patient's airways and may have a hole therein such that the flow of air at a therapeutic pressure is delivered to at least an entrance to the patient's nares. The seal-forming structure 3100 may be constructed and arranged to maintain said therapeutic pressure in the plenum chamber 3200 throughout the patient's respiratory cycle in use.

The anterior portion 3316 may be semi-rigid and/or resiliently deformable. In some examples, such as the examples shown in FIGS. 7A-11B, the anterior portion 3316 of the strap 3310 is thermoformed into a three-dimensional shape defining the plenum chamber 3200. The anterior portion 3316 of the strap 3310 may comprise a concave portion defining the plenum chamber 3200. The anterior portion 3316 may form a cavity into which the patient's nose and lips can be received.

In some examples, the seal-forming structure 3100 may be attached to the anterior portion 3316. The seal-forming structure 3100 in the example shown in FIG. 7A comprises a cushion 3130 attached to the anterior portion 3316 of the strap 3310 configured to sealingly engage the patient's face in use. The cushion 3130 may be formed from foam, an elastomeric material such as silicone or a thermoplastic elastomer, or gel, in examples. In the FIG. 7A example the seal-forming structure 3100 comprises a foam cushion 3130. In this example, both the strap 3310 and scal-forming structure 3100 are formed predominantly from foam and textile materials, which may provide for a patient interface 3000 that may be highly comfortable and which has an appearance more like sleepwear than a medical device, which may encourage patient compliance with therapy. As shown in FIG. 7A the seal-forming structure 3100 may be shaped to surround the patient's nose and mouth. The seal-forming structure 3100 may surround both the nasal airways and mouth of the patient. In other examples the seal-forming structure 3100 may surround only the nasal airways. In such an example, the patient interface 3000 may be configured to leave the patient's mouth uncovered. In some examples, the seal-forming structure 3100 may comprise a membrane configured to sealingly engage the patient's face. The membrane may be C-shaped or sickle shaped, for example, as is known in the art, and may form a pressure activated seal with the patient's face in use.

In some examples of the present technology, such as the example shown in FIGS. 8A-8B, the anterior portion 3316 of the strap 3316 may be shaped to form the seal-forming structure 3100 in addition to the plenum chamber 3200. The anterior portion 3316 of the strap 3310 may be thermoformed into a three-dimensional shape defining the plenum chamber 3200 and forming the seal-forming structure 3100. The anterior portion 3316 of the strap 3310 may comprise a concave portion defining the plenum chamber 3200, e.g. forming a cavity into which the patient's nose and lips can be received, and may further comprise a convex portion provided around the periphery of the concave portion. The convex portion may be configured to engage the patient's face to form the seal-forming structure 3100. The convex portion may be resiliently compressible to provide a cushioning function, which may be comfortable and may enable the convex portion to comply and conform to the surface geometry of the patient's face. The strap 3310 may be thicker in the convex portion than in the concave portion, for example to provide cushioning.

The examples shown in FIGS. 9A-9B, 10 and 11A-11B may have a seal-forming structure 3100 having any of the configurations described herein. In some examples the anterior portion 3316 of the strap 3310 may be shaped to form the shape of the seal-forming structure 3100. In other examples the strap 3310 may comprise a cushion 3130 inserted within layers of material forming strap 3310. Furthermore, as described above, in another form, the seal-forming structure 3100 may be attached to a posterior-facing/user-facing layer of the strap 3310.

FIG. 12 shows a further configuration in which the seal-forming structure 3100 comprises a cushion module 6100. The cushion module 6100 may be a component separate to the strap 3310 that at least partially defines the plenum chamber 3200 and comprises the seal-forming structure 3100. The cushion module 6100 may be removable and replaceable from the strap 3310. The cushion module 6100 may be formed from a single material, which may be an elastomeric material such as silicone or a TPE or a foam material. Alternatively, the cushion module 6100 may be formed from multiple materials, such as a substantially rigid material such as polycarbonate or the like, an elastomeric material such as silicone or a TPE, and/or a foam material. The strap 3310 may overlie the cushion module 6100 and provide a force to hold it in sealing position against the patient's face in use. The cushion module 6100 may comprise a chassis portion and a sealing membrane attached to the chassis portion. The chassis portion may be stiffer (either due to material thickness or material selection) than the membrane. The strap 3310 may overlie and contact/engage the chassis portion. The membrane may curl inwards around the periphery of the patient's airways so as to form a pressure-activated seal in use.

### 5.3.3.3.4 Strap Material/Construction

FIG. 7B shows an exploded view of various layers of the strap 3310 shown in FIG. 7A, and FIG. 7C shows a schematic cross section view of another example. It is to be understood that this construction or any one or more of the features, properties and variants thereof described with reference to FIG. 7B or 7C may be applied to any of the straps 3310 shown in FIGS. 7A, 8A-8B, 9A-9B, 10, 11A-11B and 12.

As shown in FIGS. 7A-7C, the strap 3310 may comprise a plurality of layers, which may be bonded to each other, for example during a thermoforming process. The strap 3310 may comprise an inwardly facing layer 3361, which may be configured to contact the patient's head in use. In some examples the inwardly facing layer 3361 is formed from a textile material to provide for a soft feel on the patient's face. The strap 3310 may also comprise a resiliently compressible inner cushion layer 3362. The inner cushion layer 3362 is formed from foam in the examples shown in FIGS. 7A-7C.

The inner cushion layer 3362 may comprise a varying thickness forming the shape of the seal-forming structure 3100 or of a portion of the strap 3310 to which the seal-forming structure 3100 is attached, as the case may be. Some or all of the other layers may comprise a uniform thickness.

The strap 3310 in some examples comprises a support member 3320. The support member 3320 may be semi-rigid (e.g. resiliently deformable but sufficiently stiff to reinforce the strap 3310 or at least the anterior portion 3316 thereof). The support member 3320 may be provided only to the anterior portion 3316 in some examples and may have a stiffness sufficient to reinforce the shape of the plenum chamber 3200 and/or the seal-forming structure 3100. The support member 3320 may be in the form of a sheet formed from a plastic material, for example. The support member 3320 may form a semi-rigid layer of the strap 3310. In some examples, the strap 3310 may comprise multiple support members 3320 or the support member 3320 may be split into two portions, such as an upper portion and a lower portion.

In the example shown in FIG. 7B, the strap 3310 comprises a substantially inextensible layer 3365, which may be formed from webbing. The substantially inextensible layer 3365 may in some examples comprise a pair of high strength portions 3366 provided adjacent to the central region 3316 on respective sides of the central region 3316. In some examples the high strength portions 3366 may be provided at or proximate to an inferior edge of the strap 3310. The high strength portions 3366 may be formed from a high strength webbing and may have a higher strength than adjacent portions of the substantially inextensible layer 3365. The substantially inextensible layer 3365 and to a further extent the high strength portions may stiffen the strap 3310, which may advantageously provide for good stability in use. In other examples, such as the example shown in FIG. 7C, the strap 3310 may not comprise a substantially inextensible layer 3365.

The strap 3310 may further comprise a resiliently compressible outer cushion layer 3363. The outer cushion layer 3363 may be formed from foam. Additionally, the strap 3310 may further comprise an outwardly facing layer 3364 configured to face away from the patient's head in use and which may define an outer surface of the strap 3310. The outwardly facing layer 3364 may be formed from a textile material which may advantageously provide for an aesthetically pleasing patient interface 3000 and one which may have an appearance of sleepwear instead of a medical device, which may help patients to comply with therapy.

At least some of the layers of the strap 3310 forming the plenum chamber 3200 and seal-forming structure 3100, e.g. in the anterior portion 3316, may be airtight to maintain the therapeutic pressure in the plenum chamber 3200. In some examples all of the layers are airtight. In some examples the inwardly facing layer 3361, inner cushion layer 3362, support member 3320, substantially inextensible layer 3365 and outer cushion layer 3363 may together be airtight. In other examples the strap 3310 may not be airtight in one or more regions and may instead allow a controlled leak to provide a vent 3400 through the material forming the strap 3310.

As shown in FIG. 7B, the strap 3310 in this example comprises a fastening portion 3332 at an end of the strap 3310 configured to attach to another portion of the strap 3310 to secure the strap 3310 around the patient's head. The fastening portion 3332 in this example comprises a hook material configured to attach to loop material on a surface of the strap 3310. In other examples ends of the strap 3310 may connect to each other by a buckle, by a magnetic connection or by another suitable connection.

In some examples, the strap 3310 may be cut to a final shape (e.g. outline) by a cutting process which both cuts out the final shape of the strap 3310 and also joins layers of the strap 3310 together at edges of the strap 3310. In one example the cutting process may be RF cutting although other suitable cutting processes for cutting textile and foam materials may also be used. In some examples the cutting process may be performed after the anterior portion 3316 of the strap 3310 is thermoformed. In such examples the cutting process may be a 3D RF cutting process since the shape of the anterior portion 3316 of the strap 3310 will be three dimensional.

### 5.3.3.3.5 Thermoformed and Non-Thermoformed Portions

As described elsewhere herein, the anterior portion 3316 of the strap 3310 may be thermoformed into a three-dimensional shape to form a plenum chamber 3200.

In some examples, further portions of the strap 3310 are also, or alternatively, thermoformed. Other portions may be substantially non-thermoformed. For example, a majority of certain portions may be non-thermoformed. In certain strap portions, only localised regions may be thermoformed.

In some examples, such as the example shown in FIG. 9B, the strap 3310 comprises lateral portions 3315 on either lateral side of the anterior portion 3316. The lateral portions 3315 in this example are substantially flat, at least in an at-rest state. It is to be understood that a strap portion is flat if it is formed from a sheet and has no predetermined curved shape. A flat strap portion will lie flat on a table at rest, for example. Other examples of the present technology, such as the examples shown in FIGS. 8A, 10 and 11A also comprise lateral portions 3315. Lateral portions 3315 on either side of the anterior portion 3316 may be flat or may have some inherent three dimensional curved shape, in examples, although are preferably soft and flexible for comfort and so that they generally do not transmit unwanted disruptive forces to the anterior portion 3316 of the strap 3310.

In some examples, such as the example shown in FIG. 10, the strap 3310 comprises a thermoformed hinge portion 3318 between the anterior portion 3316 and the lateral portions of the strap 3310. In this example, the thermoformed hinge portion 3318 comprises a debossed portion of the strap 3310. In particular, the debossed portion comprises a series of debossed lines or channels which form one or more functional hinges. The hinge portion 3318 may urge the strap 3310 to bend along a predetermined line or axis, which may be defined by the length of the hinge portion 3318. That is, if the strap 3310 experiences forces causing to bend, the hinge portion 3318 may cause the strap 3310 to do so along a predetermined line or axis defined by the hinge portion 3318, for example by being more bendable than other portions of the strap 3310 and/or by resisting bending along a direction perpendicular to the length of the hinge portion 3318. While the hinge portion 3318 is exemplified in a patient interface 3000 of a similar type to that shown in FIGS. 9A-9B, a hinge portion 3318 may also be applied to any of the patent interfaces 3000 shown in FIG. 7A-7C, 8A-8B or 11A-11B. In general, in some examples a strap 3310 of a patient interface 3000 may comprise a hinge portion 3318 formed by a thermoformed portion of the strap 3310, the hinge portion 3318 is structured to allow a portion of the strap 3310 on a first side of the hinge portion 3318 to pivot about the hinge portion 3318 with respect to a portion of the strap 3310 on a second side of the hinge portion 3318. In some examples, the hinge portion 3318 may be formed by multiple thermoformed lines, to create multiple possible lines or axes about which a strap 3310 may be encouraged to bend. In some examples a strap 3310 may comprise multiple hinge portions 3318, such as one hinge portion 3318 on each lateral side of the strap 3310 (e.g. alongside the anterior portion 3316 as in FIG. 10) or such as two or more hinge portions 3318 on each lateral side of the strap 3310.

The strap 3310 in some examples may comprise further thermoformed portions. With reference to FIGS. 11A-11D, in some examples each fastening portion 3332 comprises a plurality of thermoformed indexing portions. The indexing portions may assist the patient with adjusting the patient interface 3000, either by forming tactile guides or by helping the strap 3310 to retain a particular state of adjustment during and after adjustment. The thermoformed indexing portions may be embossed or debossed, for example. In some examples, each fastening portion 3332 may comprise at least one embossed indexing portion 3334 and at least one debossed indexing portion 3335. These are shown in FIG. 11C for example, which shows a fastening portion of a first end 3312 of the strap 3310 shown in FIGS. 11A and 11B. In some examples, one or more debossed indexing portions 3335 may comprise a hook material attached thereto and configured to form a hook-and-loop connection with the at least one embossed indexing portion 3334.

In example shown in FIGS. 11A-11D, rather than each end of the strap 3310 connecting directly to the other by a hook-and-loop connection, each of the first end 3312 and second end 3314 passes through a buckle 3333 and is secured back to itself. The buckle 3333 is shown in isolation in FIG. 11D. More or less of each end 3312 and 3314 of the strap 3310 can be pulled through the buckle 3333 to adjust the effective length (e.g. circumference) of the strap 3310. In this example. In this particular example, with reference to FIG. 11C, the debossed indexing portions 3335 comprise hook material fixed thereto. The hook material may fill the debossed indexing portions 3335. The debossing may allow the hook material to lie flush with the strap 3310, reducing bulk. Also as shown in FIG. 11C, the strap 3310 comprises five embossed indexing portions 3334 (in other examples there may be any number). The debossed indexing portions 3335, after passing through the buckle 3333, can be attached to the embossed indexing portions 3334. The embossed indexing portions 3334 may comprise a loop material or have a loop material provided thereto. In some examples, the outer layer of the strap 3310 may effectively be a loop material being formed from a textile material. The five embossed indexing portions 3334 guide the patient to attach the debossed indexing portions 3335 (and the hook material provided thereto) to a series of possible positions. For example, the patient may be guided to connected the two debossed indexing portions 3335 to the first two, second two, third two or fourth two embossed indexing portions 3334 to provide four predetermined configurations/sizes of the strap 3310. In other examples, there may be only embossed indexing portions 3334, or only debossed indexing portions 3335.

### 5.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

### 5.3.5 Decoupling Structure(s)

In one form the patient interface 3000 includes at least one decoupling structure, for example, a swivel or a ball and socket.

### 5.3.6 Connection Port

Connection port 3600 allows for connection to the air circuit 4170.

### 5.3.7 Forehead Support

In one form, the patient interface 3000 includes a forehead support 3700.

### 5.3.8 Anti-Asphyxia Valve

In one form, the patient interface 3000 includes an anti-asphyxia valve.

### 5.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.3.10 Modularity

Components of the patient interface 3000, such as a cushion module of a positioning and stabilising structure 3300 or components thereof, such as headgear straps, may come in different sizes or styles, which may correspond to different uses (e.g., mouth breathing, nasal breathing, etc.). A patient or clinician may select certain combinations of cushion module and headgear, for example, in order to optimize the effectiveness of the therapy and/or the individual patient's comfort. An example of this sort of modular design is described in PCT/SG2022/050777 filed 28 Oct. 2022, incorporated herein by reference in its entirety.

In some forms, the different styles of patient interface components may be used interchangeably with one another in order to form different styles of patient interfaces. This may be beneficial from a manufacturing prospective because a wider variety of patient interfaces may be created using fewer parts. Additionally or alternatively, the various combinations may allow a patient to change styles of patient interface without changing the every component.

Air may be delivered to the patient in one of two main ways. In one example, the patient may receive the flow of pressurized air through headgear tubes 3350 (see e.g., FIG. 3Z). This may be referred to as a “tube up” configuration and may position a connection port at the top of the patient's head. In other example, the patient may receive the flow of pressurized air through a conduit connected to the plenum chamber 3200, for example through the connection port 3600 (see e.g., FIG. 3A). This may be referred to a “tube down” configuration where the airflow conduit is positioned in front of the patient's face. Different patients may be more comfortable with one style of air delivery over the other (e.g., because of the patient's sleep style). Therefore, it may be beneficial to allow a single style of patient interface to be used in either the “tube up” or “tube down” configuration.

The patient interface 3000 may be part of a modular assembly with a variety of interchangeable components that may be swapped out by a patient and/or clinician for one or more components for a different style.

### 5.4 Rpt Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 4 cmH2O, or at least 10 cmH2O, or at least 20 cmH₂O.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 5.6 Humidifier

### 5.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 5.7 Breathing Waveforms

FIG. 6 shows a model typical breath waveform of a person while sleeping. The horizontal axis is time, and the vertical axis is respiratory flow rate.

### Respiratory Therapy Modes

Various respiratory therapy modes may be implemented by the disclosed respiratory therapy system.

### 5.8 Glossary

For the purposes of the present technology disclosure, in certain forms of the present technology, one or more of the following definitions may apply. In other forms of the present technology, alternative definitions may apply.

### 5.8.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜0.001 atm). In this specification, unless otherwise stated, pressure is given in units of cmH₂O.

Respiratory Pressure Therapy: The application of a supply of air to an entrance to the airways at a treatment pressure that is typically positive with respect to atmosphere.

5.8.1.1 Materials & their Properties

Hardness: Refers to durometer or indentation hardness, which is a material property measured by indentation of an indentor (e.g., as measured in accordance with ASTM D2240).
‘Soft’ materials may include silicone or thermo-plastic elastomer (TPE), and may, e.g. readily deform under finger pressure.
‘Hard’ materials may include polycarbonate, polypropylene, and may not e.g. readily deform under finger pressure.

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 5.8.1.2 Mechanics

### Axes:

a. Neutral axis: An axis in the cross-section of a beam or plate along which there are no longitudinal stresses or strains.
b. Longitudinal axis: An axis extending along the length of a shape. The axis generally passes through a center of the shape.
c. Circumferential axis: An axis oriented perpendicularly with respect to the longitudinal axis. The axis may be specifically present in pipes, tubes, cylinders, or similar shapes with a circular and/or elliptical cross section.

Deformation: The process where the original geometry of a member changes when subjected to forces, e.g. a force in a direction with respect to an axis. The process may include stretching or compressing, bending and, twisting.

Elasticity: The ability of a material to return to its original geometry after deformation.

Floppy structure or component: A structure or component that will change shape, e.g. bend, when caused to support its own weight, within a relatively short period of time such as 1 second.

Resilience: Ability of a material to absorb energy when deformed elastically and to release the energy upon unloading.

Resilient: Will release substantially all of the energy when unloaded. Includes e.g. certain silicones, and thermoplastic elastomers.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Viscous: The ability of a material to resist flow.

Visco-elasticity: The ability of a material to display both elastic and viscous behaviour in deformation.

Yield: The situation when a material can no longer return back to its original geometry after deformation.

### 5.8.1.3 Structural Elements

Compression member: A structural element that resists compression forces.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Membrane: Membrane will be taken to mean a typically thin element that has, preferably, substantially no resistance to bending, but has resistance to being stretched.

Tie (noun): A structure designed to resist tension.

### Thin Structures:

a. Beams,
   i. A beam may be relatively long in one dimension compared to the other two dimensions such that the smaller dimensions are comparatively thin compared to the long dimension
b. Membranes,
   i. Relatively long in two dimensions, with one thin dimension. Readily deforms in response to bending forces. Resists being stretched, (might also resist compression).
c. Plates & Shells
   i. These may be relatively long in two directions, with one thin dimension. They may have bending, tensile, and/or compressive stiffness.

### Thick Structures: Solids

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

### 5.8.2 Anatomy

### 5.8.2.1 Anatomy of the Face

Ala: the external outer wall or “wing” of each nostril (plural: alar)

Alar angle: An angle formed between the ala of each nostril.

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius): The lip extending between the subnasale and the mouth.

Lip, upper (labrale superius): The lip extending between the mouth and the supramenton.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

Anatomy of the skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 5.8.3 Patient Interface

Anti-asphyxia valve (AAV): The component or sub-assembly of a mask system that, by opening to atmosphere in a failsafe manner, reduces the risk of excessive CO2 rebreathing by a patient.

Headgear: Headgear will be taken to mean a form of positioning and stabilising structure designed to hold a device, e.g., a mask, on a head.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.8.4 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 5.8.4.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 5.8.4.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical-topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 5.8.4.3 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 5.9 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means+/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A patient interface for treatment of sleep disordered breathing, the patient interface comprising:
a plenum chamber pressurisable to a therapeutic pressure of at least 4 cmH2O above ambient air pressure, said plenum chamber including a plenum chamber inlet port sized and structured to receive a flow of air at the therapeutic pressure for breathing by a patient;
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a positioning and stabilising structure configured to provide a force to hold the seal-forming structure in a therapeutically effective position on the patient's head, the positioning and stabilising structure comprising a strap configured to be arranged, in use, so that an anterior portion of the strap is positioned anterior to the patient's face in use;
a vent to allow a continuous flow of gases exhaled by the patient from an interior of the plenum chamber to ambient, said vent being configured to maintain the therapeutic pressure in the plenum chamber in use;
wherein the anterior portion of the strap is shaped to form the plenum chamber;
wherein the seal-forming structure is formed by or provided to the anterior portion of the strap.

2. The patient interface of claim 1, wherein the anterior portion of the strap is thermoformed into a three-dimensional shape defining the plenum chamber.

3. The patient interface of claim 3, wherein the anterior portion of the strap comprises a concave portion defining the plenum chamber.

4. The patient interface of claim 1, wherein the seal-forming structure is attached to the anterior portion of the strap.

5. The patient interface of claim 1, wherein the seal-forming structure comprises a cushion attached to the anterior portion of the strap configured to sealingly engage the patient's face in use.

6. The patient interface of claim 5, wherein the cushion is formed from foam.

7. The patient interface of claim 1, wherein the anterior portion of the strap is shaped to form the seal-forming structure.

8. The patient interface of claim 7, wherein the anterior portion of the strap is thermoformed into a three-dimensional shape defining the plenum chamber and forming the seal-forming structure.

9. The patient interface of claim 8, wherein the anterior portion of the strap comprises a concave portion defining the plenum chamber and a convex portion provided around a periphery of the concave portion configured to engage the patient's face to form the seal-forming structure.

10. The patient interface of claim 9, wherein the strap is thicker in the convex portion than in the concave portion.

11. The patient interface of claim 1, wherein the strap comprises an inwardly facing layer configured to contact the patient's head in use and being formed from a textile material.

12. The patient interface of claim 1, wherein the strap comprises a resiliently compressible inner cushion layer.

13. The patient interface of claim 12, wherein the inner cushion layer is formed from foam.

14. The patient interface of claim 12, wherein the inner cushion layer comprises a varying thickness forming the shape of the seal-forming structure or of a portion of the strap to which the seal-forming structure is attached.

15. The patient interface of claim 1, wherein the strap comprises a semi-rigid support member.

16. The patient interface of claim 1, wherein the strap comprises a substantially inextensible layer.

17. The patient interface of claim 16, wherein the substantially inextensible layer comprises a pair of high strength portions provided adjacent to the anterior portion on respective sides of the anterior portion and having a higher strength than adjacent portions of the substantially inextensible layer.

18. The patient interface of claim 17, wherein the high strength portions are provided at or proximate to an inferior edge of the strap.

19. The patient interface of claim 1, wherein the strap comprises a resiliently compressible outer cushion layer.

20. The patient interface of claim 19, wherein the outer cushion layer is formed from foam.

21. The patient interface of claim 20, wherein the strap comprises an outwardly facing layer configured to face away from the patient's head in use and being formed from a textile material.

22. The patient interface of claim 1, wherein the strap comprises lateral portions on either lateral side of the anterior portion, the lateral portions being substantially flat at least at rest.

23. The patient interface of claim 22, wherein the strap comprises a thermoformed hinge portion between the anterior portion and the lateral portions of the strap.

24. The patient interface of claim 1, wherein the strap comprises a fastening portion at an end of the strap configured to attach to another portion of the strap to secure the strap around the patient's head, wherein each fastening portion comprises a plurality of thermoformed indexing portions.

25. The patient interface of claim 24, wherein each fastening portion comprises at least one embossed indexing portion and at least one debossed indexing portion.

26. The patient interface of claim 25, wherein the at least one debossed indexing portion comprises a hook material attached thereto and configured to form a hook-and-loop connection with the at least one embossed indexing portion.
